(19) United States
(12) Patent Application Publication
(10) Pub. No.: US 2022/0384061 A1
(43) Pub. Date: Dec. 1, 2022

(54) SYSTEM AND METHOD FOR SECURE HEALTHCARE PROFESSIONAL COMMUNICATION

(71) Applicant: Optiks Solutions, Inc., Piscalaway, NJ (US)

(72) Inventors: Anupam NANDWANA, Piscataway, NJ (US); Edward VAZ, Piscataway, NJ (US)

(21) Appl. No.: 17/751,931

(22) Filed: May 24, 2022

Related U.S. Application Data
(60) Provisional application No. 63/192,572, filed on May 25, 2021.

(51) Int. Cl. G16H 80/00; H04W 4/14; G16H 10/60

(52) U.S. Cl. CPC G16H 80/00; H04W 4/14; G16H 10/60

(57) ABSTRACT

A system and method for secure healthcare professional communication with patients that employs a variety of different messaging modalities. Examples of suitable messaging modalities that may be used with the present invention include but are not limited to SMS, Facebook Messenger, WhatsApp, WeChat and other such modalities. Optionally and preferably, such modalities may be employed with the present invention but without requiring additional plug-ins, software and/or other apps to the communication device of the healthcare professional and/or the patient.

### FIELD OF THE INVENTION

The present invention, in at least some embodiments, is of a system and method for secure healthcare professional communication, and in particular, to such a system and method securely employing a variety of different messaging modalities with a variety of different roles, including without limitation healthcare professionals, office staff, external support staff or patients or caregivers thereof.

### BACKGROUND OF THE INVENTION

Communication with healthcare professionals is very important. Such individuals communicate with others in a variety of different roles, such as other healthcare professionals, office staff, external support staff or patients or caregivers thereof. Security and confidentiality are both important to maintain patient privacy, even when communication is not occurring directly with patients. However convenience is also important; individuals today have become accustomed to the convenience of direct messaging and/or voice calls through smart phones and other personal communication devices. Such convenience may permit more rapid communication which is also important in today's high pressure world of healthcare. Combining both convenience and security can be quite difficult.

### BRIEF SUMMARY OF THE INVENTION

The present invention in at least some embodiments, is of a system and method for secure healthcare professional communication with individuals in a variety of different roles that employs a variety of different messaging modalities, while maintaining security and convenience across all of the different roles and modalities. Examples of individuals with different roles with whom such communication may be performed include but are not limited to healthcare professionals, office staff, external support staff, or patients or caregivers thereof. External support staff may include staff at diagnostic laboratories, other medical centers, and staff at providers of goods and services to healthcare professionals, including without limitation sales staff, pharmacists, medical device specialists, scientists and the like.

Examples of suitable messaging modalities that may be used with the present invention include but are not limited to SMS, MMS, Facebook Messenger, WhatsApp, WeChat, iMessage, Apple Business Chat, Telegram, Signal, Skype message and other such modalities. Optionally and preferably, such modalities may be employed with the present invention but without requiring additional plug-ins, software and/or other apps to the communication device of the healthcare professional and/or the patient. The term “patient” as used herein may refer to a patient receiving care and/or a caregiver.

According to at least some embodiments, there is provided a system for supporting secure healthcare professional communication, comprising a computer network, a sending user computational device, a server and a recipient user computational device, wherein said sending user computational device, said server and said recipient user computational device communicate through said computer network; wherein each of said sending user computational device and said recipient user computational device comprise a messaging modality, wherein said messaging modality comprises a modality addressed through a telephone number and a social media modality; wherein said sending user computational device creates a message according to a template determined according to at least one policy; wherein said message is sent from said sending user computational device to said server and is addressed according to said telephone number or said social media address; and wherein said server relays said message according to said telephone number or said address to said recipient user computational device; wherein said telephone number or said address of said sending user computational device and said recipient user computational device are each masked.

Optionally said sending user computational device comprises an external support staff user computational device and said recipient user computational device comprises a healthcare professional computational device, wherein the device is controllable by a healthcare professional. Optionally said external support staff comprises staff at diagnostic laboratories, other medical centers, and staff at providers of goods and services to healthcare professionals, including without limitation sales staff, pharmacists, medical device specialists and scientists; wherein said sending user computational device is associated with said external support staff and wherein said policy is enforced according to an external support staff organization. Optionally said telephone number is associated with a mobile device connected to or identified with an external support staff member or an external support staff organization. Optionally said telephone number is associated with a mobile device connected to or identified with a healthcare professional or a healthcare professional office.

Optionally said message comprises an SMS message or a MMS message. Optionally said social media address comprises one or more of Facebook Messenger, WhatsApp, WeChat, iMessage, Apple Business Chat, Telegram, Signal, or Skype; and wherein said message is sent and/or received through said social media channel.

According to at least some embodiments, there is provided a method for increasing reliability of messaging communication through a messaging modality with a HCP (healthcare provider), wherein the messaging modality is executed through a computational device by the HCP, the method comprising: receiving a request for medical information through the messaging modality; generating, under control of one or more computer systems, a message that provides the medical information and that includes message content, and recipient identification data for the HCP; reviewing the message content for compliance by a computer system; sending the message to the HCP if compliance has been established; confirming a delivery of the message to the HCP; and generating an audit trail that is configured to create a historical record corresponding to said message.

Optionally said reviewing the message content for compliance comprises analyzing the message content by a NLP algorithm and preventing the message from being sent if non-compliant content is detected. Optionally said non-compliant content comprises one or more stop words. Optionally said non-compliant content comprises content that is not appropriate for the HCP, according to an analysis of a license or other certification held by the HCP.

Optionally, the method further comprises confirming a delivery of the message to the HCP; or alternatively, upon receiving indication of delivery failure of the message, sending the message via a different redundant communication modality. Optionally, the method further comprises validating the message and/or a response from the HCP according to a contact detail and/or a parameter of the HCP.

Optionally, said parameter comprises one or both of a license of the HCP or an office address corresponding to a license of the HCP. Optionally, the audit trail includes message timestamp data for the message, delivery attempt data including corresponding timestamp data, response data, identification data for the HCP and/or user identification data. Optionally, transmitting the message through the messaging modality and the review for non-compliant content are compliant with healthcare and/or pharmaceutical service specific requirements when operated in combination.

According to at least some embodiments, there is provided a method for increasing reliability of messaging communication through a messaging modality with a patient, wherein the messaging modality is executed through a computational device by the patient, the method comprising: initiating transmission of patient-specific medical information through the messaging modality to the computational device by one or more of an HCP, HCP staff member and/or external support staff member; generating, under control of one or more computer systems, a message that provides the patient specific medical information and that includes message content, and recipient identification data for the patient; reviewing the message content for compliance by a computer system; sending the message to the patient if compliance has been established; confirming a delivery of the message to the patient; and generating an audit trail that is configured to create a historical record corresponding to said message.

Optionally, said reviewing the message content for compliance comprises analyzing the message content by a NLP algorithm and preventing the message from being sent if non-compliant content is detected. Optionally, said non-compliant content comprises one or more stop words. Optionally, said non-compliant content comprises content that is not medically relevant to the patient. Optionally, said non-compliant content comprises content that includes patient specific medical details that are protected under medical regulations.

Optionally, affirmative consent for such content to be transmitted through the messaging modality has not been provided by the patient. Optionally, the method further comprises confirming a delivery of the message to the patient; or alternatively, upon receiving indication of delivery failure of the message, sending the message via a different redundant communication modality. Optionally, the method further comprises validating the message and/or a response from the patient according to a contact detail of the patient.

Optionally, the audit trail includes message timestamp data for the message, delivery attempt data including corresponding timestamp data, response data, identification data for the patient and/or user identification data for the transmitting user. Optionally, transmitting the message through the messaging modality and the review for non-compliant content are compliant with healthcare and/or pharmaceutical service specific requirements when operated in combination.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Unless otherwise defined, all technical and scientific terms used herein have the same meaning as commonly understood by one of ordinary skill in the art to which this invention belongs. The materials, methods, and examples provided herein are illustrative only and not intended to be limiting.

An algorithm as described herein may refer to any series of functions, steps, one or more methods or one or more processes, for example for performing data analysis.

Implementation of the apparatuses, devices, methods and systems of the present disclosure involve performing or completing certain selected tasks or steps manually, automatically, or a combination thereof. Specifically, several selected steps can be implemented by hardware or by software on an operating system, of a firmware, and/or a combination thereof. For example, as hardware, selected steps of at least some embodiments of the disclosure can be implemented as a chip or circuit (e.g., ASIC). As software, selected steps of at least some embodiments of the disclosure can be implemented as a number of software instructions being executed by a computer (e.g., a processor of the computer) using an operating system. In any case, selected steps of methods of at least some embodiments of the disclosure can be described as being performed by a processor, such as a computing platform for executing a plurality of instructions. The processor is configured to execute a predefined set of operations in response to receiving a corresponding instruction selected from a predefined native instruction set of codes.

Software (e.g., an application, computer instructions) which is configured to perform (or cause to be performed) certain functionality may also be referred to as a “module” for performing that functionality, and also may be referred to a “processor” for performing such functionality. Thus, processor, according to some embodiments, may be a hardware component, or, according to some embodiments, a software component.

Further to this end, in some embodiments: a processor may also be referred to as a module; in some embodiments, a processor may comprise one or more modules; in some embodiments, a module may comprise computer instructions—which can be a set of instructions, an application, software—which are operable on a computational device (e.g., a processor) to cause the computational device to conduct and/or achieve one or more specific functionality. Some embodiments are described with regard to a “computer,” a “computer network,” and/or a “computer operational on a computer network.” It is noted that any device featuring a processor (which may be referred to as “data processor”; “pre-processor” may also be referred to as “processor”) and the ability to execute one or more instructions may be described as a computer, a computational device, and a processor (e.g., see above), including but not limited to a personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, a pager, and/or a similar device. Two or more of such devices in communication with each other may be a “computer network.”

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIG. 1 shows a non-limiting, exemplary system for healthcare communication;

FIG. 2 relates to a non-limiting, exemplary method for contact management;

FIG. 3 shows a non-limiting, exemplary method for initiating communication with a healthcare professional;

FIG. 4 shows a non-limiting, exemplary method for initiating communication with a staff member of an office of a healthcare professional;

FIG. 5 shows a non-limiting, exemplary method for initiating communication with a contact that was not previously known;

FIG. 6 shows a non-limiting, exemplary method for communication with a healthcare professional;

FIG. 7 shows a non-limiting, exemplary method for communication with a staff member of an office of a healthcare professional;

FIG. 8 shows a non-limiting, exemplary method for communication with a previously unknown contact;

FIG. 9 shows a non-limiting, exemplary method for sending a message with an attachment to a HCP contact and/or a HCP staff contact;

FIG. 10 shows a non-limiting, exemplary method for an opt-in to initiate communication through the system;

FIG. 11 shows a non-limiting, exemplary method for an opt-out to further communication through the system;

FIG. 12 shows a non-limiting, exemplary method for outbound voice communication from a healthcare professional to a patient;

FIG. 13 shows a non-limiting, exemplary method for inbound voice communication from a patient to a healthcare professional;

FIG. 14 relates to a non-limiting, exemplary method for calling a recipient who is not already present in the CRM;

FIG. 15 shows a non-limiting, exemplary system for signature capture;

FIG. 16 relates to a non-limiting, exemplary method for sending a MIR template to a contact;

FIG. 17 relates to a non-limiting, exemplary method for scanning a code, such as a QR code for initiating communication by a recipient;

FIG. 18 shows an exemplary, non-limiting process for transmitting the previously described code, such as a QR code for example, to the recipient;

FIG. 19 shows an exemplary, non-limiting process for handling an out of office message;

FIG. 20 shows a non-limiting, exemplary method for communication between an external support staff member and a recipient, who could be a healthcare professional (HCP) or HCP staff for example, using a specific example of a messaging service;

FIG. 21 shows a non-limiting, exemplary architecture for content moderation;

FIG. 22 relates to a non-limiting, exemplary method for content moderation of transmitted messages through the system as described herein;

FIG. 23 relates to a non-limiting, exemplary architecture for bot communication;

FIG. 24 relates to a non-limiting, exemplary method for bot communication;

FIG. 25 shows a non-limiting, exemplary method for communication logging into a database;

FIG. 26 relates to a non-limiting, exemplary architecture for a video call;

FIG. 27 relates to a non-limiting, exemplary method for a video call;

FIG. 28 relates to a non-limiting, exemplary architecture for scheduling a call;

FIG. 29 relates to a non-limiting, exemplary flow for scheduling a call;

FIG. 30 relates to a non-limiting, exemplary architecture for short URL creation and redirection;

FIG. 31 relates to a non-limiting, exemplary flow for short URL creation and redirection;

FIG. 32 relates to a non-limiting, exemplary architecture for a group chat;

FIG. 33 relates to a non-limiting, exemplary flow for a group chat;

FIG. 34 relates to a non-limiting, exemplary architecture for short code messaging;

FIG. 35 relates to a non-limiting, exemplary flow for short code messaging;

FIG. 36 relates to a non-limiting, exemplary method for handling a MIR request;

FIG. 37 relates to a non-limiting, exemplary method for handling a sample request;

FIG. 38 shows a system for supporting communication with regard to multiple modalities, while FIG. 39 shows a non-limiting, exemplary method for communication through such a system; and

FIGS. 40 and 41 show the system of FIG. 38 in more detail.

### DESCRIPTION OF AT LEAST SOME EMBODIMENTS

Digital communications in regulated industries require additional management and control than such communication in non-regulated industries. For example, there are strict rules regarding communication between providers of medical services, termed herein “healthcare providers” (HCP), and/or their staff, and companies that support the HCP with products and/or services, such as pharmaceutical companies for example. HCPs wish to receive information about these products and/or services on demand, or at least with a minimum of delay. Companies wish to market their products and/or services to HCP. However, these communications are regulated by the relevant healthcare regulation body in that country, such as the FDA (Food and Drug Administration) in the US.

In a regulated area such as healthcare, a company may need to provide medically appropriate information, but also wishes to sell a product or service. Regulations in the US require that the provision of medically appropriate information be separated from the provision of commercial information. For example, scientifically and/or medically trained personnel at a pharmaceutical company may provide information about a drug, including therapeutic effects, side effects and other medical information; while a sales representative may provide commercial information about the drug. In a digitally connected world, technology is required to separate these types of communications and to support provision of information through different communication channels.

HCPs may also wish to communicate with a company through channels that previously were not used for medical business communication, such as messaging apps such as WhatsApp, SMS messages and other messaging modalities. Such channels may support a faster response time, and hence more efficient provision of information, but also need careful management to avoid transgressing a regulation and/or providing incorrect information. Consistency of messaging across multiple channels is also important. In addition, communications and interactions across multiple channels need to be logged for compliance purposes. Therefore, communication is both faster when distributed across multiple touchpoints and with multiple parties participating (such as different groups of personnel at a drug company, as appropriate for medical vs commercial communications), yet also needs to be managed in a centralized manner, for compliance logging, and to ensure information is accurate and consistent.

Different communication modalities also support faster provision of customized information. Given a greater understanding of, and research into, multi-drug interactions, effects of aging on dosage levels, treatment of comorbidities and other specialist medical information, HCPs now require information that is customized to a particular patient's circumstances. As patient compliance is also understood to be important, customized communication with or on behalf of a patient is also important. The methods and systems as described herein may be applied to communication with a patient, whether from an external support staff member, and/or an HCP or an HCP staff member.

Content moderation may be applied by an AI system as described herein, to ensure that messages are compliant with regulations, such as healthcare regulations. Such content moderation may be combined with the messaging systems as described herein, to ensure that appropriate content is sent to the appropriate recipient. For example, these systems may ensure that medical information is only sent to a HCP or HCP staff. These systems may also ensure that physical samples of a medical product are only sent to a HCP who has the appropriate license to receive that product. These systems may therefore reduce the risk of information and/or a product being sent to a contact who does not have the requisite credentials, such as a license, to receive that information and/or product.

Additionally, such systems may also protect the privacy of HCPs and/or personnel at companies who provide products and/or services, by masking the contact details (such as a telephone number, email address and/or other information), while still enabling messages to be correctly and quickly routed to the recipient.

In combination, these systems not only reduce risk but also increase the reliability of messaging transmission. Information may be sent faster and with a greater degree of personalization and/or customization, yet still remain compliant with healthcare regulations.

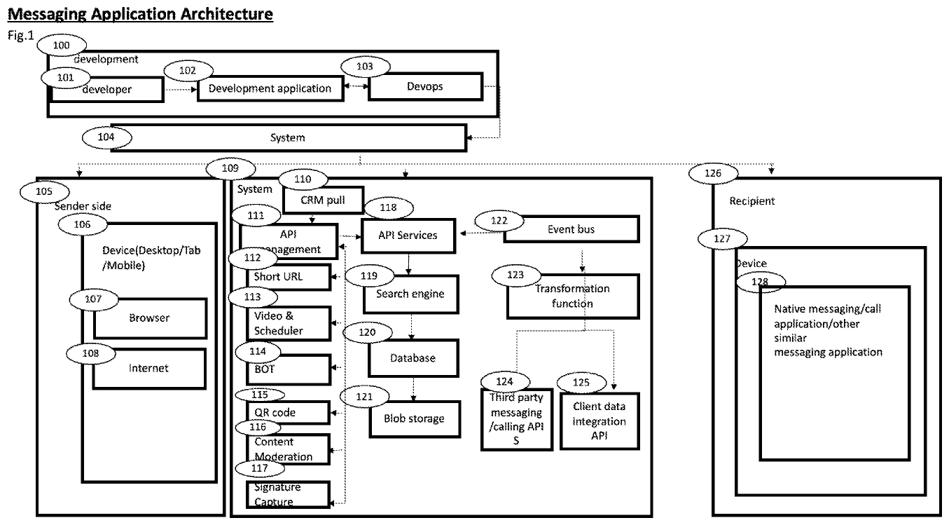

FIG. 1 shows a non-limiting, exemplary system for secure healthcare communication. As shown, the system features a developer environment 100 for developing one or more applications for an application system 104. Developer environment 100 preferably features a developer 101, interacting with a development application 102 and supported by one or more developer tools 103. The output from developer environment 100 supports the addition of functionalities to application system 104.

Application system 104 supports communication between a sender side 105 and a recipient side 126, through a server side 109. Preferably no additional software is installed at sender side 105 and/or recipient side 126, although one or more software modules are installed at server side 109 to support communication.

Sender side 105 preferably comprises any suitable computational device 106 that is capable of operating a message application 107. Optionally message application 107 is any suitable, standard, off the shelf message application as described herein, or alternatively may comprise any suitable browser based and/or mobile phone operating system based application. Message application 107 is preferably able to communication with server side 109 through for example a regular messaging channel connection, including without limitation a messaging app identifier and/or a mobile device telephone number. Message application 107 may also comprise a web browser as shown. Communication may be supported by any suitable communication channel, including without limitation any type of computational network such as the internet, separate cellular network communication and so forth, shown as internet 108.

Server side 109 preferably exchanges messages with message application 107, for example to send to another such message application, of the same or a different type, such as application 128 operated by a recipient computational device 126 as shown. Again, any suitable messaging channel connection may be used to connect application 128 and server side 109, including without limitation a messaging app identifier and/or a mobile device telephone number. Preferably both message application 107 and application 128 are multi-purpose such applications, rather than being installed for the specific purpose of communication through server side 109. Such application may comprise messaging applications, calling (voice communication) applications and so forth. Non-limiting examples of such messaging applications include WhatsApp, Telegram, Signal, Facebook Messenger, WeChat, Viber, Line, Google messaging, Instagram for business, Apple chat, native SMS application functionality, native or live chat functionality and the like. In each case, rather than connecting directly through the servers or communication channels normally associated with each such application, both message application 107 and application 128 connect with a phone number, address or other identifier which appears to be a normal identifier for each such message application 107 or 128, but instead are directed to connect through server side 109, which mediates the connection.

Server side 109 comprises an API management 111, which receives various types of communication, including but not limited to such exchanged messages. API management 111 then determines the correct API to which the communication is to be passed, and then passes the communication to one or more 111API services 118. 111API services 118 may pass the communication directly to an external API (not shown), and/or may pass the communication to a search engine 119 and/or an 119event bus 122.

Search engine 119 may comprise one or more AI models and/or be configured as an AI engine as described herein. Search engine 119 may determine which external API is to receive the communication according to one or more models and/or machine learning algorithms. For example, search engine 119 may analyze the content of the communication and determine that it relates to an emergency, as well as whether the communication is being received from a patient or other subject in the emergency, or from a healthcare provider. Search engine 119 may also determine that the communication relates to a request for information, to a connection to a particular healthcare provider or patient, to a request for an appointment and so forth. Search engine 119 may retrieve required information, such as the information needed to make a further connection and/or to answer the request for information, from a database 120. Such requests may then be logged in a physical storage, such as a blob storage 121.

Turning now to event bus 122, the communication may be further passed to a 120transformation function 123, which determines whether the communication is to be passed to one or more 121third party messaging APIs 120 and/or to a client data integration API 125. One or more third party messaging APIs 124 may for example support communication with message application 107 and application 128, for example as one or more of WhatsApp, Telegram, Signal, Facebook Messenger, WeChat, native SMS application functionality and the like.

Client data integration API 125 preferably supports communication with an external client data source and/or software. The role of “client” may for example be performed by any suitable stakeholder, including but not limited to healthcare professionals, office staff, and/or external support staff. External support staff may include staff at diagnostic laboratories, other medical centers, and staff at providers of goods and services to healthcare professionals, including without limitation sales staff, pharmacists, medical device specialists, scientists and the like.

Server side 109 may also comprise a URL shortening module 112, for easier transmission of links to chats, video calls, information and so forth. Server side 109 may also comprise a video call management module 113, which enables video calls to be scheduled and then supports communication through such video calls. Server side 109 may also comprise a bot 114 for supporting automated communication, for example through a messaging function as described herein.

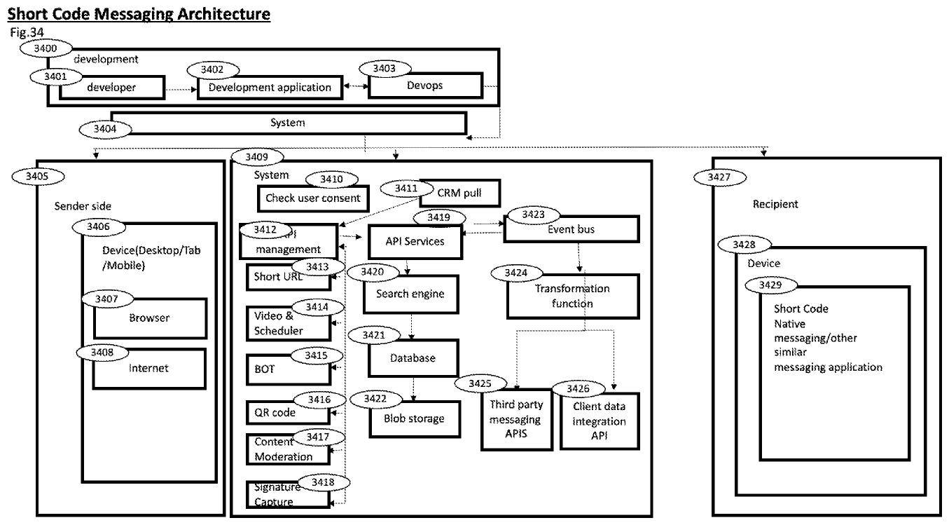

FIG. 34 relates to a non-limiting, exemplary architecture for short code messaging, which is similar to that of FIG. 1, but is specifically directed to mobile telephony. In particular, a mobile telephony device 3428 is able to operate a messaging app 3429, which is able to provide Short Code Native messaging and/or another similar messaging application.

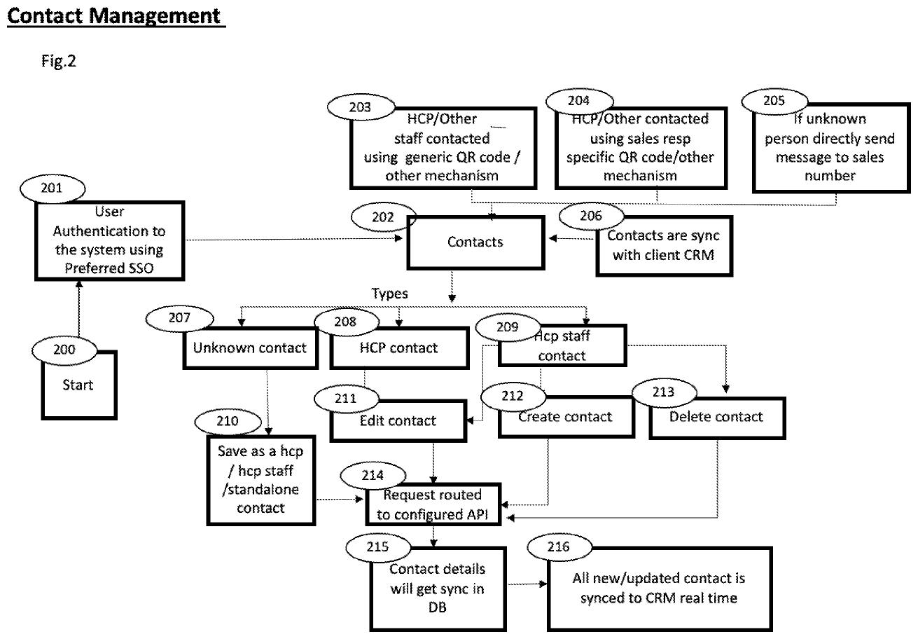

FIG. 2 relates to a non-limiting, exemplary method for contact management. The method may be operated by the system of FIG. 1 as a non-limiting example. As shown, the method 200 begins with user authentication to the system, for example using SSO (single sign on) technology. Next, the user is able to review contacts 202, optionally filtering them by type. Some non-limiting examples of types of contacts to be filtered, and then optionally updated and/or reviewed, include an unknown contact 207, which may then be saved as a HCP (healthcare provider) or HCP staff at 210. A HCP contact 208 may be edited (updated, changed, created and/or deleted) at 207. A HCP staff contact 209 may be edited at 211, created at 212 and/or deleted at 213. Optionally, creation of a new HCP contact 208 may be performed by saving/associating unknown telephone numbers and/or message modality addresses (if not telephone numbers) that are flowing into the system. The contact may be a HCP or a non-HCP contact.

After any of these actions 210-213 are performed, then at 214, the request for the results of any of these contact management actions to be saved in the database is preferably sent to the configuration API. The contacts database is then updated at 215. The CRM is also preferably synchronized at 216.

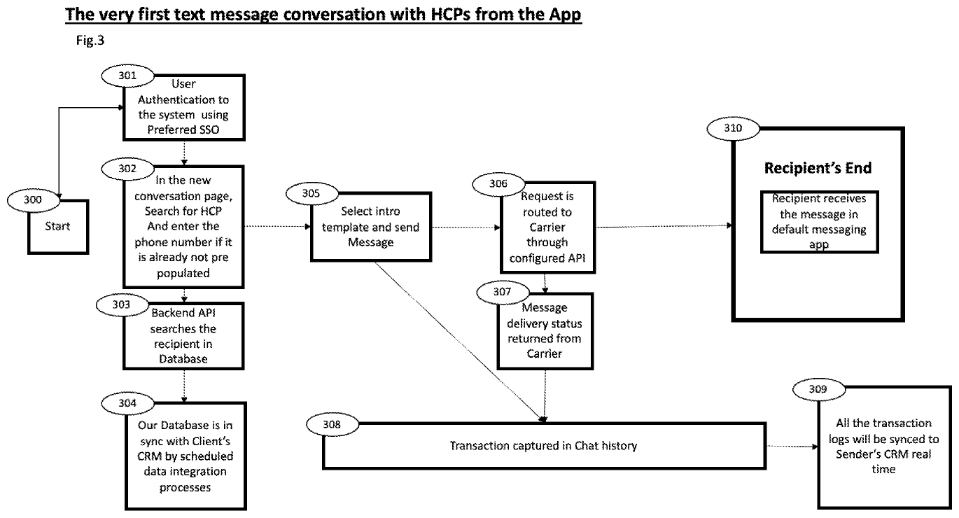

FIG. 3 shows a non-limiting, exemplary method for initiating communication with a healthcare professional. As shown, a healthcare provider, office staff member, external support staff member or other user initiates communication at 300, for example by logging into a software interface. For the purpose of description only, the user is assumed to be an external support staff member attempting to contact an HCP (healthcare professional). The user in this non-limiting example is the sender of a message and the HCP is the recipient. The user is then invited to authenticate at 301, for example by using an SSO (single sign-on) method, which may be performed through an existing email, social media or other user account. At 302, the user may be shown a landing page and/or may employ a voice command to indicate which healthcare professional is to be contacted. If the phone number or other identifier of that healthcare professional is not available, then preferably the user enters it. Although the phone number is a preferred identifier for the healthcare professional, optionally a social media account identifier or other identifier may be used. Where suitable, such an identifier may be substituted for “phone number” when the latter is discussed herein.

At 303, the backend API receives the request and searches for the correct healthcare provider information, for example in the previously described server database. The healthcare provider information may include for example contact details of the healthcare provider to be contacted, including but not limited to phone number and/or social media contact information. Optionally such contact information is for a “cut-out” communication modality, in which the private contact details of the HCP are kept private, while contact information that the server supports is provided. At 304, the database is preferably synchronized with the CRM (customer relationship management) database of the external support staff company or organization by scheduled data integration processes. For example, such an synchronization may be performed through the client data integration API of FIG. 1 (not shown).

At 305, an introduction or other suitable template is selected, for initiating a new message, for example to the selected HCP as recipient. If this is the first time that the user has contacted the recipient using this message modality, or optionally any modality, then preferably an introduction template is selected. The message is then sent. At 306, the request to send the message is routed as necessary to transmit it. For example, for an SMS, the request is preferably routed to a cellular network communication provider, such as a carrier, through the previously described configured API.

At 307, the delivery status is returned from the carrier or other message communication provider. Optionally, if the delivery status is a failed delivery, then the message may be sent through a different messaging modality and/or repeated through the same messaging modality. This option applies to all messaging methods and systems as shown herein, and for all embodiments and Figures as shown herein. The message transaction is preferably captured in a chat history of communication between that authenticated user and the specific recipient at 308.

At 309, the recipient receives the message in their default messaging app for that type of communication, such as an SMS message app on a mobile communication device for example. Preferably the default is set in advance and is stored in the previously described CRM.

At 309, all the transaction logs are synchronized to the customer's CRM in real time or in a batch process.

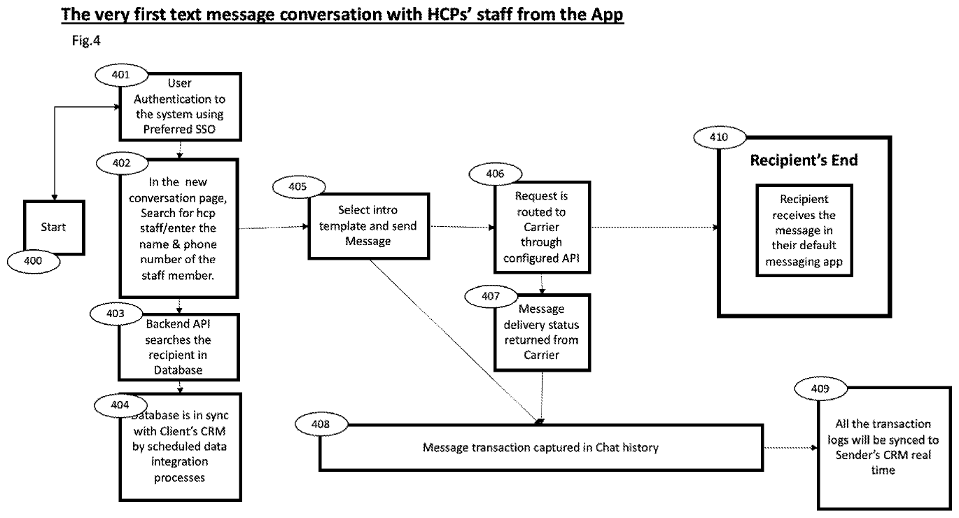

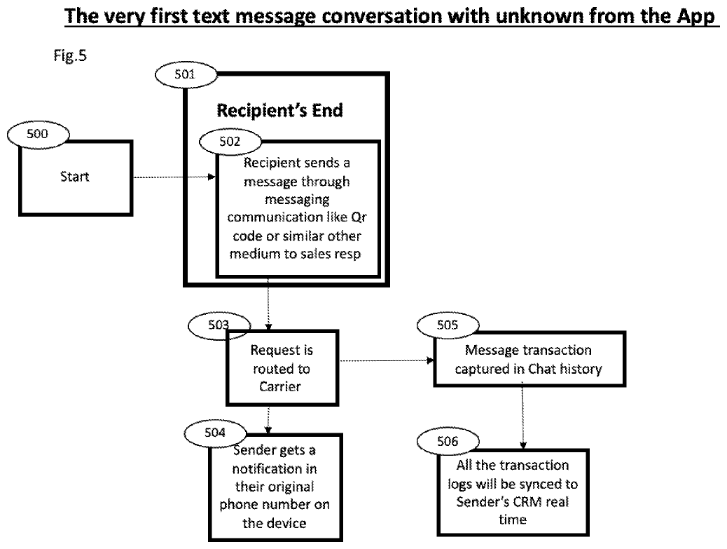

FIG. 4 shows a non-limiting, exemplary method for initiating communication with a staff member of an office of a healthcare professional by an external support staff member. For example, optionally the method shown in FIG. 3 is performed with a healthcare professional as recipient, while the staff interactions may be performed through the method shown with regard to this drawing. The external support staff member initiates communication at 400, for example by logging into a software interface, and then by authenticating at 401, as previously described.

At 402, the user may be shown a landing page and/or may employ a voice command to indicate which member of the office staff of a healthcare professional is to be contacted. If the phone number or other identifier of that office staff member is not available, then preferably the user enters it. Although the phone number is a preferred identifier for the office staff member, optionally a social media account identifier or other identifier may be used. Where suitable, such an identifier may be substituted for “phone number” when the latter is discussed herein.

At 403, the backend API receives the request and searches for the correct office staff member information, for example in the previously described server database. The office staff member information may include for example contact details of the staff member to be contacted, including but not limited to phone number and/or social media contact information. Optionally such contact information is for a “cut-out” communication modality, in which the private contact details of the staff member are kept private, while contact information that the server supports is provided. At 404, the database is preferably synchronized with the CRM (customer relationship management) database of the external support staff company or organization by scheduled data integration processes. For example, such an synchronization may be performed through the client data integration API of FIG. 1 (not shown).

At 405, an introduction or other suitable template is selected, for initiating a new message, for example to the selected office staff member as recipient. If this is the first time that the user has contacted the recipient using this message modality, or optionally any modality, then preferably an introduction template is selected. The message is then sent. At 406, the request to send the message is routed as necessary to transmit it. For example, for an SMS, the request is preferably routed to a cellular network communication provider, such as a carrier, through the previously described configured API.

At 407, the delivery status is returned from the carrier or other message communication provider. The message transaction is preferably captured in a chat history of communication between that authenticated user and the specific recipient at 408.

At 409, all the transaction logs are synchronized to the customer's CRM in real time or in a batch process.

At 410, the recipient receives the message in their default messaging app for that type of communication, such as an SMS message app on a mobile communication device for example. Preferably the default is set in advance and is stored in the previously described CRM.

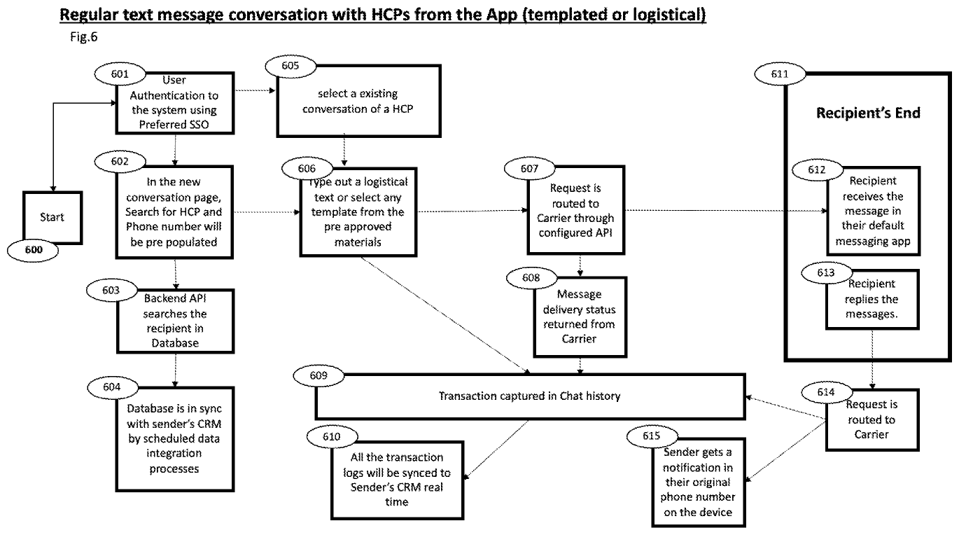

FIG. 5 shows a non-limiting, exemplary method for initiating communication with a contact that was not previously known. In this non-limiting example, the recipient designated in other flows, such as HCP or HCP staff member, initiates the process by sending a contact to a sales representative, or other representative or personnel of a company or organization. As shown, the process begins at 500. At 501, a recipient decides to initiate communication as described above. At 502, the recipient sends a message through a messaging communication modality. For example, the communication may be initiated by scanning a QR code as described herein. The message is sent a sales representative or other personnel.

At 503, the request to send the message is routed as necessary to transmit it. For example, for an SMS, the request is preferably routed to a cellular network communication provider, such as a carrier, through the previously described configured API.

At 504, the recipient of the message (the sender in other flows as described herein) receives the message from the unknown contact. The message transaction is preferably captured in a chat history of communication between the recipient and sender at 505.

At 506, all the transaction logs are synchronized to the customer's CRM in real time or in a batch process.

FIG. 6 shows a non-limiting, exemplary method for communication between an external support staff member and a recipient, who could be a healthcare professional for example. In this non-limiting example, the conversation is assumed to be between an HCP and an external support staff member. Preferably, this method is employed after communication is established between the healthcare professional and the external support staff member, whether generally or for this specific communication modality. Optionally, stages 600-604 are similar or identical to stages 300-304 of FIG. 3. An existing conversation with an HCP may optionally be selected to begin, at 604.

At 606, the external support staff member determines the message structure and content, for example from one or more templates, and/or as free-form text, or a combination thereof. For example and without limitation if the healthcare professional has previously requested assistance with a particular good or service, and/or is requesting information, then a suitable template may be selected, for example to request that the healthcare professional call the external support staff member, login to a secure portal, or otherwise perform some action. Alternatively or additionally, the external support staff member may transmit information directly in the message. Optionally the external support staff member may request the HCP to perform some other action such as a booking an appointment.

At 607, the request to send the message is routed as necessary to transmit it as previously described. In this non-limiting example, the message is an SMS message and the request is routed to a suitable carrier.

At 608, the delivery status is returned from the carrier or other message communication provider. The message transaction is preferably captured in a chat history of communication between that authenticated user and the specific recipient at 609.

At 610, all the transaction logs are synchronized to the customer's CRM in real time or in a batch process as previously described.

At 611, the recipient receives the message, for example in their default messaging app for that type of communication, such as an SMS message app on a mobile communication device for example, shown at 612. In this non-limiting example, the recipient is assumed to be a healthcare professional. Again, preferably the default is set in advance and is stored in the previously described CRM. At 613 the recipient replies, whether because the message content requested a reply or independently. At 614, the request is routed through the carrier for an SMS message. At 615, the user receives a notification, which is routed to that user according to the previously entered phone number or other identifier. The notification may be sent by the backend as an SMS message, or alternatively may be sent as some other type of message, according to the user contact configuration.

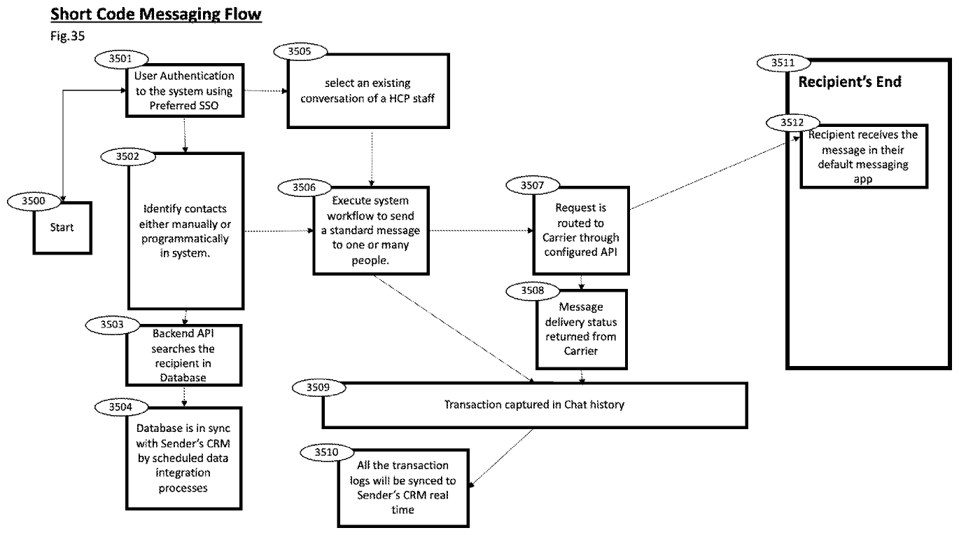

FIG. 35 relates to a non-limiting, exemplary flow for short code messaging through mobile telephony.

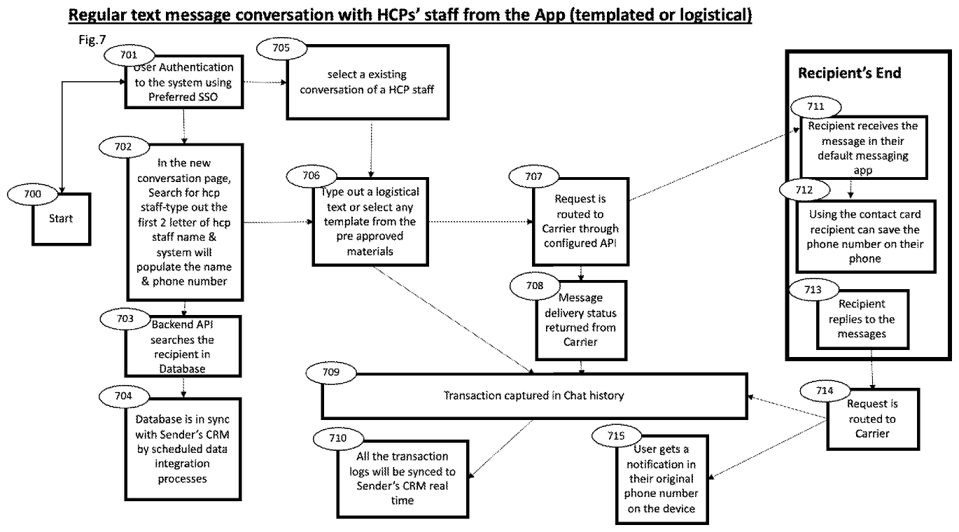

FIG. 7 shows a non-limiting, exemplary method for communication between a staff member of an office of a healthcare professional and a patient or an external support staff member. In this non-limiting example, the communication is between the staff member of the healthcare professional and the external support staff member. Optionally, stages 700-704 are similar or identical to stages 400-404 of FIG. 4.

Optionally, at 705, the message may be sent as part of an existing conversation with a HCP staff contact, for example as part of a message thread or in reply to an already sent message.

At 706, the external support staff member determines the message structure and content, for example from one or more templates, and/or as free-form text, or a combination thereof. For example and without limitation if the external support staff member wishes to transmit information or alternatively wants the HCP staff member to contact them, then a suitable template may be selected, for example to request that the HCP staff member call the external support staff member, the HCP and/or the office; login to a secure portal, or otherwise perform some action. Alternatively or additionally, the external support staff member may send information directly in the message.

Stages 707-714 may be similar or identical to stages 607-614 of FIG. 6. At 715, optionally the external support staff member receives a notification, which is routed to that user according to the previously entered phone number or other identifier. The notification may be sent by the backend as an SMS message, or alternatively may be sent as some other type of message, according to the user contact configuration. If there are multiple recipients of the notification, then optionally different messaging modalities may be used.

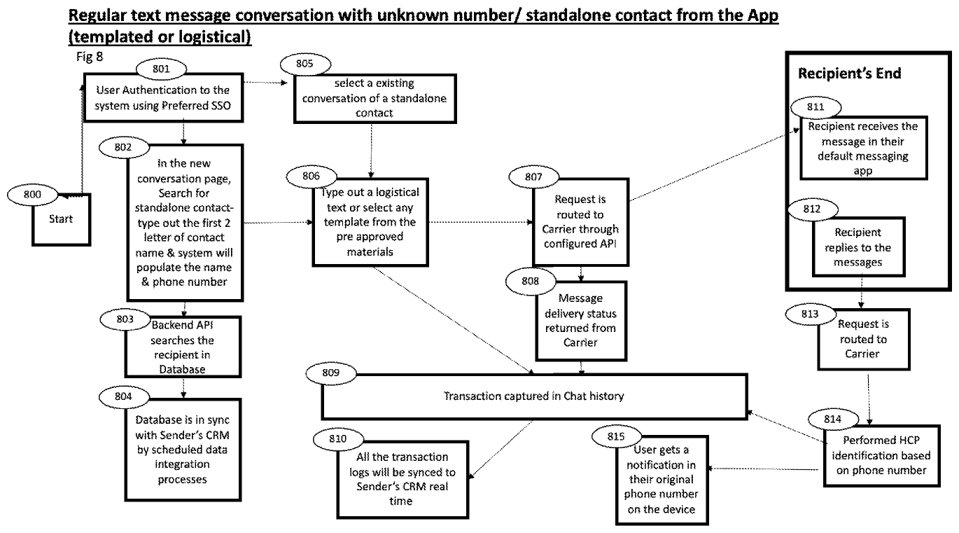

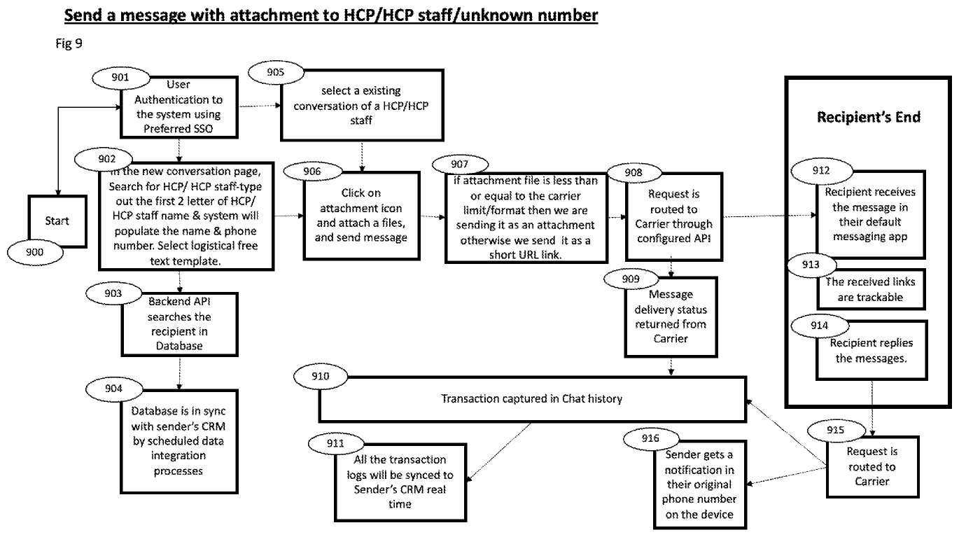

FIG. 8 shows a non-limiting, exemplary method for communication with a previously unknown contact. Optionally, stages 800-804 are similar or identical to stages 700-704 of FIG. 7.

Optionally, at 805, the message may be sent as part of an existing conversation with the unknown contact, for example as part of a message thread or in reply to an already sent message. Optionally contact information is logged as soon as a conversation is initiated and is stored in a database and/or CRM system as described herein.

At 806, the external support staff member determines the message structure and content, for example from one or more templates, and/or as free-form text, or a combination thereof. For example and without limitation if the external support staff member wishes to transmit information or alternatively wants the unknown contact to contact them, then a suitable template may be selected, for example to request that the unknown contact call the external support staff member; login to a secure portal, or otherwise perform some action. Alternatively or additionally, the external support staff member may send information directly in the message. Optionally such information is limited in content because the contact is unknown, and hence it is not possible to determine whether certain content is permitted to be sent.

Stages 807-811 may be similar or identical to stages 707-711 of FIG. 7. At 812, the recipient replies the message, for example using the messaging modality as previously described. At 813, the request to send the message is routed as necessary to transmit it. For example, for an SMS, the request is preferably routed to a cellular network communication provider, such as a carrier, through the previously described configured API.

At 814, optionally an identification process is performed to identify the unknown contact. For example, the telephone number may be associated with a HCP or a HCP staff member, or with a medical organization such as a hospital for example.

At 815, optionally the external support staff member receives a notification, which is routed to that user according to the previously entered phone number or other identifier. The notification may be sent by the backend as an SMS message, or alternatively may be sent as some other type of message, according to the user contact configuration. If there are multiple recipients of the notification, then optionally different messaging modalities may be used.

FIG. 9 shows a non-limiting, exemplary method for sending a message with an attachment to a HCP contact and/or a HCP staff contact. As shown, a healthcare provider, office staff member, external support staff member or other user initiates communication at 900, for example by logging into a software interface. For the purpose of description only, the user is assumed to be an external support staff member attempting to contact an HCP. The user in this non-limiting example is the sender of a message and the HCP is the recipient. The user is then invited to authenticate at 901, for example by using a SSO method, which may be performed through an existing email, social media or other user account.

At 902, if a new conversation is to be started (that is, a conversation or message that is not based on a previously sent message), then the user enters the new conversation page. Optionally, a previous conversation may be defined as one that is performed according to a single messaging modality, although optionally and alternatively, a previous conversation may also combine a plurality of communication modalities, including a plurality of messaging modalities and/or other communication modalities. In that page, the user searches for a contact from a HCP/HCP staff. As a non-limiting example, the user may enter the first two letters of the name of the contact, and/or of the name of the office or organization of the contact. The system may then populate the name, phone number and optionally other contact information such as an email address and/or cellular phone number. The user then preferably selects the logistical free text template. A logistical free text template is a template which enables a freely written or created text message/attachment to be sent from the new conversion screen, or from a screen related to an existing conversation.

Next, at 903, the backend API receives the request and searches for the contact information, for example in the previously described server database. The contact information may include for example contact details, including but not limited to phone number and/or social media contact information. Optionally such contact information is for a “cut-out” communication modality, in which private contact details are kept private, while contact information that the server supports is provided. At 904, the database is preferably synchronized with the CRM (customer relationship management) database of the external support staff company or organization by scheduled data integration processes. For example, such a synchronization may be performed through the client data integration API of FIG. 1 (not shown).

Optionally, at 905, the message may be sent as part of an existing conversation with a HCP staff contact, for example as part of a message thread or in reply to an already sent message.

At 906, the user preferably selects one or more message files and attaches the file(s) to the message. For example and without limitation, the file(s) may be attached to an SMS message. At 907, the size of the attachment(s) is considered. If the size is below the limitation for a particular message channel and/or carrier, then the attachments are sent attached to the message. Otherwise, the attachment(s) may be saved to an online cloud service, in which case a short URL link is sent with the message instead.

At 908, the request to send the message is routed as necessary to transmit it. For example, for an SMS, the request is preferably routed to a cellular network communication provider, such as a carrier, through the previously described configured API.

At 909, the delivery status is returned from the carrier or other message communication provider. The message transaction is preferably captured in a chat history of communication between that authenticated user and the specific recipient at 910. At 911, all the transaction logs are synchronized to the customer's CRM in real time or in a batch process.

At 912, the recipient receives the message in their default messaging app for that type of communication, such as an SMS message app on a mobile communication device for example. Preferably the default is set in advance and is stored in the previously described CRM. At 913, if the attachment(s) are instead provided through one or more links, the recipient clicks on the one or more links to receive the attachment(s). This action is preferably trackable. Otherwise, the recipient is preferably able to view the attachment(s) directly from, or by downloading the attachment(s) from, the message. At 914, optionally the recipient replies to the message. If so, then the request is routed through the carrier at 915.

At 916, optionally the user receives a notification, which is routed to that user according to the previously entered phone number or other identifier. The notification may be sent by the backend as an SMS message, or alternatively may be sent as some other type of message, according to the user contact configuration. If there are multiple recipients of the notification, then optionally different messaging modalities may be used.

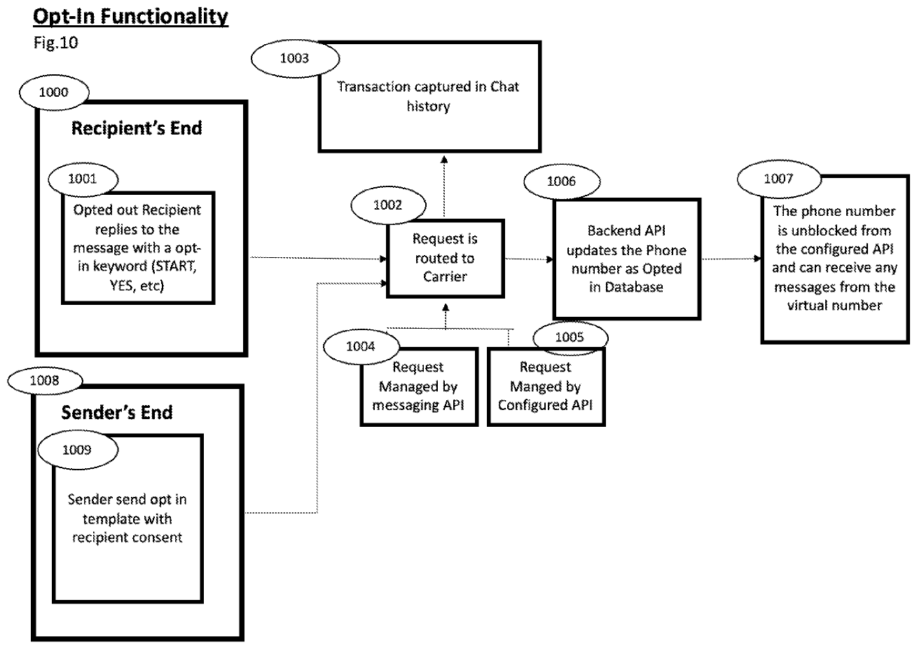

FIG. 10 shows a non-limiting, exemplary method for an opt-in to initiate communication through the system. Such a method may be performed if for example the recipient opted-out accidentally and/or if the recipient has now decided to opt back in to such communication. In this non-limiting example, the opt-in is requested by a recipient, which is assumed to be a device connected to a patient and/or an HCP or HCP staff member. The sender is assumed to be a healthcare professional and/or staff member, and/or an external support staff member. At 1000, the recipient receives a message as previously described. At 1001, the recipient replies to the message sent as previously described with an opt-in request, which may for example comprise a suitable keyword (START, YES, etc).

At 1002, the opt-in request message is routed as necessary to transmit it as previously described. In this non-limiting example, the message is an SMS message and the request is routed to a suitable carrier. The carrier then routes it to the backend server, as the virtual number is associated with the backend server. The message transaction is preferably captured in a chat history of communication between that authenticated user and the specific recipient at 1003.

At 1004, the request is managed by the messaging API. At 1005, the request is managed by the configured API.

At 1006, the Backend API updates the phone number as an “opt in” in the relevant database, so that further requests from the sender to send a message to that phone number and/or other communication modality may be supported. For example, at 1007, the opted out phone number is unblocked from the carrier's end and is now able to receive any SMS messages from the virtual number.

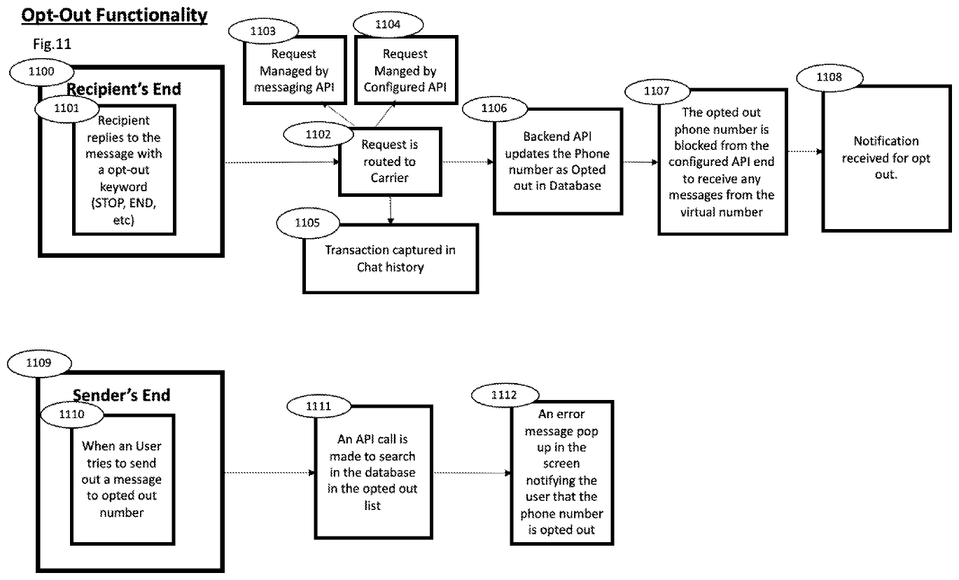

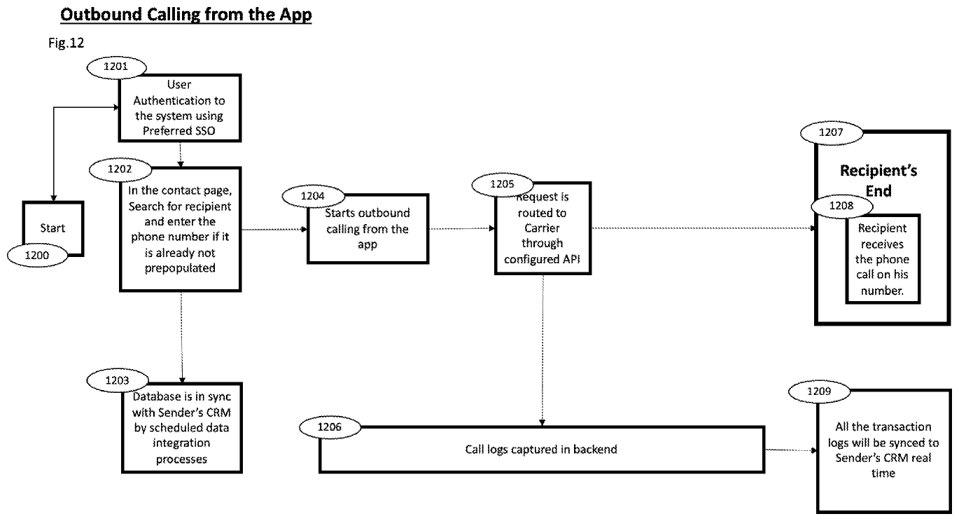

FIG. 11 shows a non-limiting, exemplary method for an opt-out to further communication through the system. In this non-limiting example, the opt-out is requested by a recipient, which is assumed to be device connected to a HCP and/or a patient. The sender is assumed to be an external support staff member and/or a healthcare professional and/or staff member. At 1100, the recipient receives the message, and replies to the message sent as previously described with an opt-out request at 1101, which may for example comprise a suitable keyword (STOP, END, etc).

At 1102, the opt-out request message is routed as necessary to transmit it as previously described. In this non-limiting example, the message is an SMS message and the request is routed to a suitable carrier. The carrier then routes it to the backend server, as the virtual number is associated with the backend server. At 1103, the request is managed by the messaging API. At 1104, the request is managed by the configured API. The message transaction is preferably captured in a chat history of communication between that authenticated user and the specific recipient at 1105.

At 1106, the Backend API updates the phone number as an “opt out” in the relevant database, so that further requests from the sender to send a message to that phone number and/or other communication modality are preferably blocked. For example, at 1107, the opted out phone number is blocked from the carrier's end to receive any SMS messages from the virtual number. The opt-out may be confirmed at 1108. At 1109, the sender and/or others in the organization are notified of the opt out.

At 1110, the sender attempts to send a message to the opted-out identifier, such as an opted-out phone number in this non-limiting example. At 1111, before the message is sent, preferably an API call is made to search in the database in the opted out list. Preferably such a search is performed before any message is sent, for example for any of the message transmission methods as described herein, to avoid inadvertently transmitting a message to an opted-out identifier. At 1112, since an attempt is being made to send the message to an opted-out identifier, preferably an error message is provided. For example, the error message may pop up in the screen notifying the user (sender) that the phone number is opted out.

FIG. 12 shows a non-limiting, exemplary method for outbound voice communication from an external support staff member to an HCP or an HCP staff member. The external support staff member may be a sales representative for a pharmaceutical or other medical supply company. Optionally, stages 1200-1203 are similar or identical to stages 300-304 of FIG. 3.

At 1204, the user, which in this example is a healthcare professional, preferably starts outbound calling from the app to a particular patient (recipient) phone number and/or synchronous voice communication modality. The latter may include but is not limited to WhatsApp, Skype, WeChat and the like. The voice communication modality may also comprise video where supported by the communication modality selected.

At 1205, the request is routed to a suitable carrier and/or other communication channel through a configured API. For example, the carrier may comprise a telephone and/or cellular network provider. Other suitable communication channels may be selected according to the communication modality, including but not limited to WhatsApp, Skype, WeChat and the like. At 1206, preferably the call logs are captured at the backend, including with regard to initiated calls that are not answered, as well as those in which a conversation occurs.

At the recipient 1207, the recipient receives the synchronous voice communication session initiation request, such that a synchronous voice communication session may begin at 1208. For example, for a call to a phone number of the recipient, the synchronous voice communication session may be a telephone call. For other voice communication modalities as described herein, the session may be a voice and/or video call.

At 1209, all the transaction logs are synchronized to the customer's CRM in real time or in a batch process as previously described.

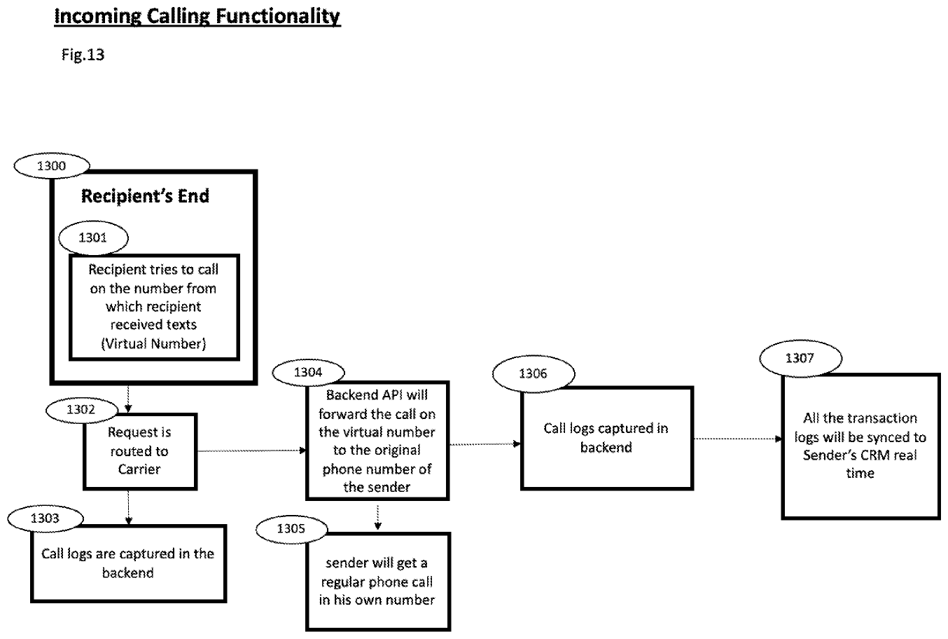

FIG. 13 shows a non-limiting, exemplary method for inbound communication from a patient to a healthcare professional. A similar method could be used for communication between an external support staff member and an HCP or an HCP staff member. In this example, the recipient, who is assumed to be a patient, attempts to initiate a synchronous voice communication session according to the previously received session initiation request. For example, at the recipient's side, the recipient may wish to initiate a telephone or other voice conversation, based on a previously received message, at 1300. At 1301, the recipient may attempt to call back the telephone number from the request, which may be an SMS message or other message service message. The telephone number from the request from the healthcare professional is a virtual number, such that preferably a direct telephone call is not performed.

Instead, at 1302, the request is routed to a carrier for a telephone call, as in this example the recipient dials the number as for any other type of telephone call. At 1303, the call is preferably logged, even for unsuccessful connection attempts as previously described.

At 1304, the Backend API preferably forwards the call from the virtual number to the original phone number of the user, which in this example is a healthcare professional. At 1305, the user receives a regular telephone call on their number. At 1306, the call may be logged again at the backend. At 1307, all the transaction logs are synchronized to the customer's CRM in real time or in a batch process as previously described.

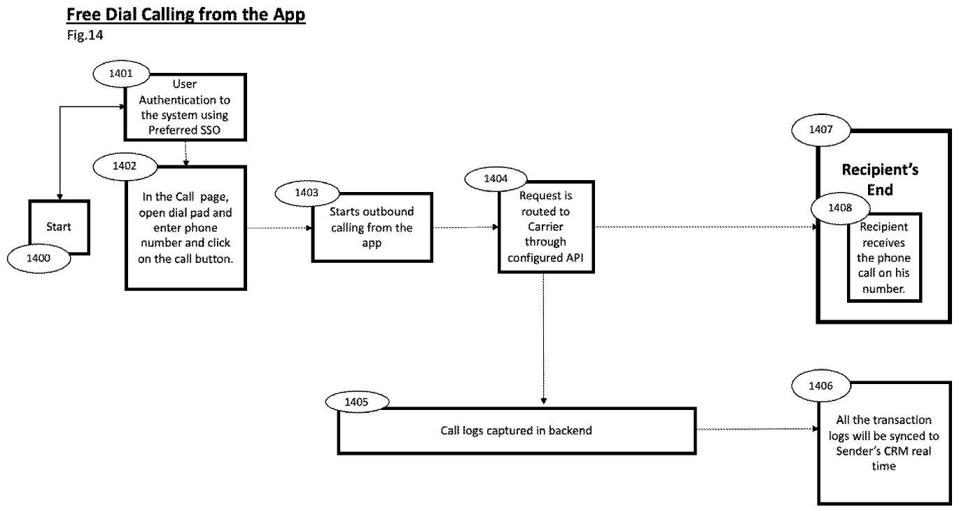

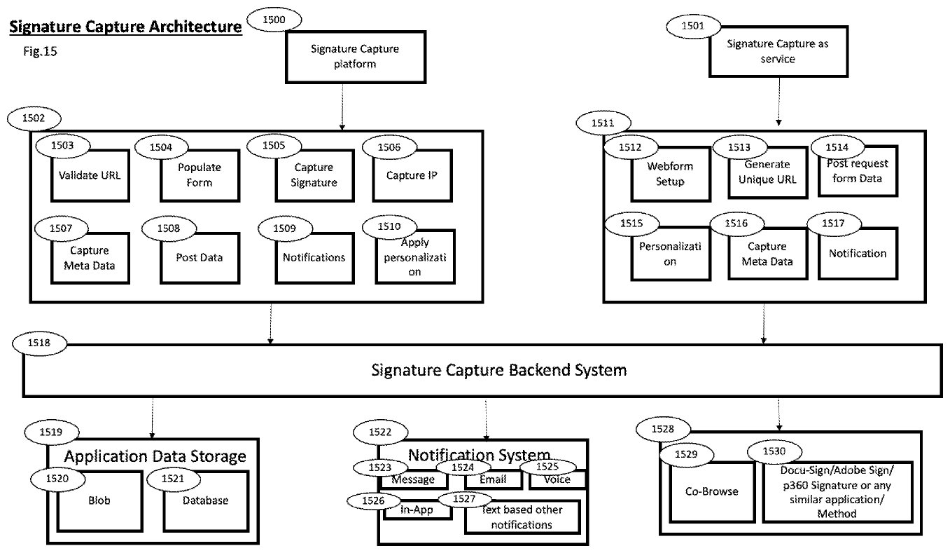

FIG. 14 relates to a non-limiting, exemplary method for calling a recipient who is not already present in the CRM. This method may be used for example for a new contact number. While the description relates to an outbound telephone call, other types of voice calls may also be performed. A similar method may be performed for sending a message to a contact that is not in the CRM for the first time as well.

As shown, the method begins at 1400 when the user decides to initiate such a call and activates the app or other interface to the system as described herein. At 1401, the user authenticates to the system using SSO or another login process as described herein. At 1402, the user navigates to the call page or screen. In the call page or screen, the user opens the dial pad and enters the phone number. The number may be entered by manipulating the on-screen GUI (graphical user interface) buttons for the app, by entering numbers through the keyboard of the device, and/or by copying and pasting the number onto the screen. The user then clicks on the call button.

At 1403, the app causes the outbound call to be initiated. At 1404, the call is routed through the correct carrier, including a cellular provider, a landline provider, any VoIP (voice over IP) or other voice call service.

At 1405, preferably the call logs are captured at the backend, including with regard to initiated calls that are not answered, as well as those in which a conversation occurs. At 1406, all the transaction logs are synchronized to the customer's CRM in real time or in a batch process as previously described.

At 1407, the recipient receives the synchronous voice communication session initiation request, such that a synchronous voice communication session may begin. For example, for a call to a phone number of the recipient, the synchronous voice communication session may be a telephone call. For other voice communication modalities as described herein, the session may be a voice and/or video call. The recipient may choose to connect at 1408.

FIG. 15 shows a non-limiting, exemplary system for signature capture. A signature of a HCP or a HCP staff member may be required for authorization of a request, for example for medical information and/or a sample as described herein. As shown, a signature capture platform 1500 features a plurality of components 1502, including but not limited to a validate URL module 1503, which preferably validates the URL for which signature capture is requested. A populate form module 1504 may then populate the form which is to be signed and/or otherwise to provide the content for which assent or acknowledgement is agreed by the act of signing. A capture IP module 1505 preferably determines the IP address of the computational device which is being used to sign the form or other content, for example for security reasons and/or to be included with the signed form. A capture meta data module 1506 may also capture meta data relating to the computational device being used to sign the form or other content, and/or in relation to the time of day, date, day of the week and so forth. Such meta data may be captured for security reasons and/or to be included with the signed form. A post data module 1508 may provide this data and/or other information to a signature capture backend system 1518. Optionally, a notifications module 1509 notifies a requester of the signature, the signing entity or both, or other entities, upon signing of the form or other content. A personalization module 1510 optionally applies personalization to the form or other content, the act of signing and/or the notification.

Optionally, the signature may be captured through a signature capture service 1501. Such a service may be operated through a plurality of components 1511 as shown, including without limitation a web form setup module 1512, for entering the necessary information for the form. A unique URL generator 1513 may create a unique URL for this instance of the form at this time of signing. A post request form data module 1514, a personalization module 1515, a meta data capture module 1516 and a notification module 1517 may operate as previously described.

Signature capture backend system 1518 then preferably sends the transmitted information, which may include one or more of verification of the signature, the form information or other content, meta data, any security verification such as IP address verification, and so forth, to an application data storage 1519. Such information may then be stored in a blob form 1520 and/or a database 1521.

A notification system 1522 optionally receives notifications from the previously described notification modules, and then sends a notification through a suitable messaging modality, including but not limited to a message service 1523, an email service 1524, a voice service 1525, an in-app service 1526 and/or another type of text based notification 1527.

An API service 1528 may feature access to other services, including but not limited to co-browsing service 1529 and a further signature verification method 1530.

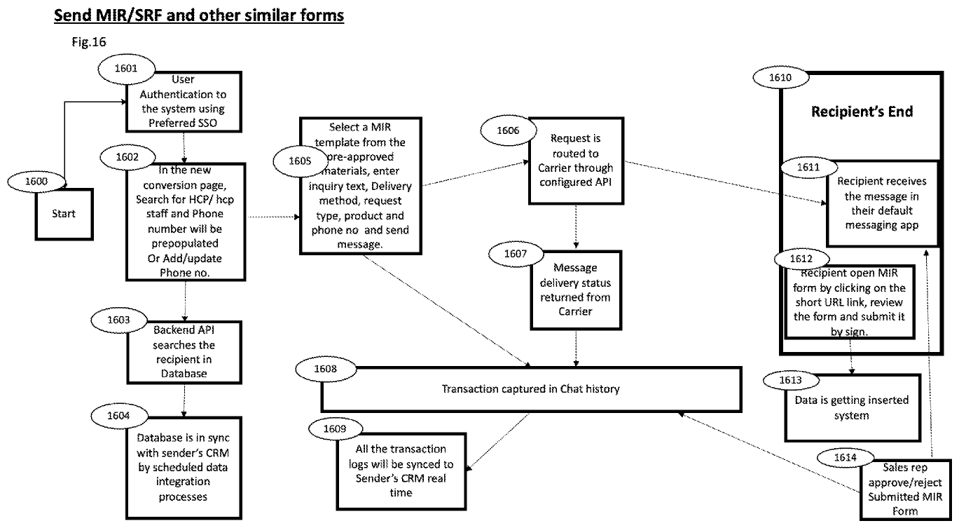

FIG. 16 relates to a non-limiting, exemplary method for sending a MIR template to a contact. The contact may for example be a HCP and/or a HCP staff member contact. Steps 1600-1604 may be performed as described with regard to steps 900-904 of FIG. 7. A MIR is a Medical Enquiry Request. Such a form may be used when particular medical, biological or other regulated information is requested, for example and without limitation with regard to side effects of a drug. Only certain personnel of a distribution and/or pharmaceutical manufacturing company are allowed by law to answer such a request for information. A similar process may be followed for a request to receive a physical sample, such as a SRF.

At 1605, the user selects a MIR template from the pre-approved materials. The user then enters a question and sends the SMS. The SMS or other message preferably includes a link to the MIR form, more preferably as a short URL as described herein.

Steps 1606-1609 may be performed as described with regard to steps 908-911.

Next, at the recipient side, at 1610, the recipient receives the message at 1611, preferably in their default messaging app. The type of default messaging app and the contact details for that app are preferably stored in the CRM, with regard to contact information for the recipient.

At 1612, the recipient opens the MIR form by clicking on the short URL link, reviews the form and submits it as signed. At 1613, the submitted information is received at the CRM and optionally at any other necessary database, such as a database required for regulatory compliance for example. At 1614, the sales representative, or other personnel of the company or organization who sent the message, approves or rejects the MIR submission. For example, the MIR submission may request information which the HCP or support staff is not entitled to receive, for example due to medical licensing requirements. The signature capture and sender approval process is to ensure the authenticity of HCP and HCP staff. A similar process may be performed for requesting a physical sample of a medical device, pharmaceutical or other medical supplies. These forms are preferably generated at run time.

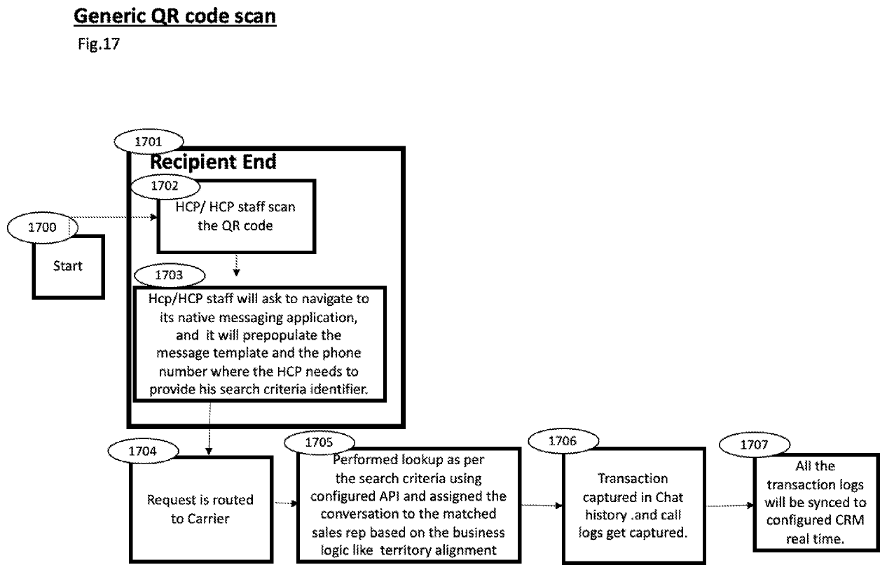

FIG. 17 relates to a non-limiting, exemplary method for scanning a code, such as a QR code, for initiating communication by a recipient. Once a QR code is scanned, the system identifies the HCP or HCP staff, for example according to location detection, zip code, territories, NPID inputs, and/or on the basis of inputs provided by scanner The NPID is a unique identifier for HCP identification. This information is preferably matched against the conversations existing in the system to associate the contact. If the contact is not identified, it is preferably entered as an unknown number into the system for the sales representative or other personnel to check and follow up.

The process starts at 1700, when a recipient receives or has access to a QR code at 1701. For this non-limiting example, the recipient is assumed to be a HCP or HCP staff, although a similar process may be used for patients or other contacts.

At 1702, the recipient scans the QR code, for example with a smart phone or other internet-connected camera. At 1703, the recipient is asked to navigate to its native messaging application. The native application then prepopulates the SMS template, including with regard to the phone number. Such a template may require the recipient to provide a NPID or other identifier. The recipient may then send an SMS or other message with the required information.

At 1703, the message is routed to the correct carrier as described herein. At 1705, an NPID lookup is performed using the configured API. The conversation is assigned to the matched sales representative based on ZIP territory alignment or other information.

At 1706, preferably the message history is captured by the backend system as previously described. At 1707, all the transaction logs are synchronized to the customer's CRM in real time or in a batch process as previously described.

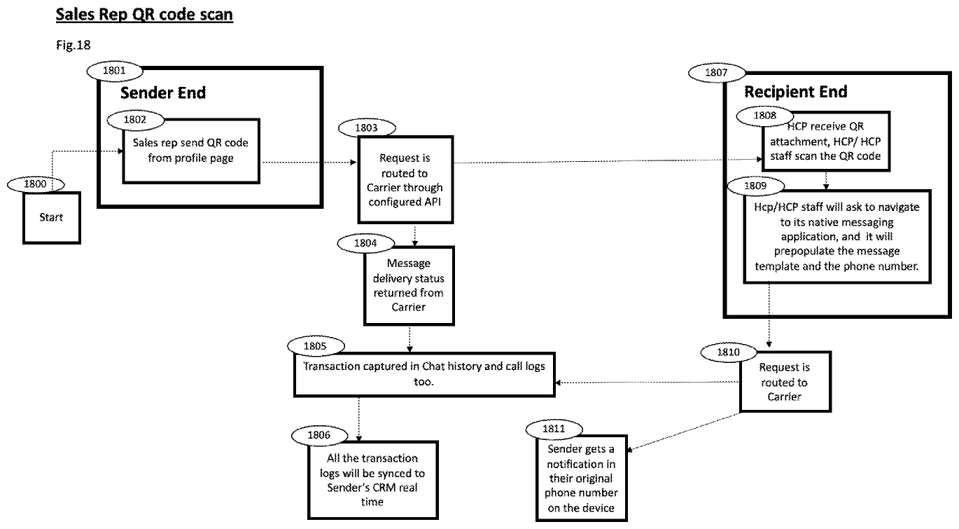

FIG. 18 shows an exemplary, non-limiting process for transmitting the previously described code, such as a QR code for example, to the recipient. The sender may be a sales representative or other representative of an external organization, who wishes to contact an HCP or HCP staff. The process begins at 1800, when the sender logs into the system as previously described. At 1801, the sender generates the code, such as the QR code for example. At 1802 the sender transmits the QR code from the profile page.

At 1803, the request is routed to the correct carrier for the type of message service that the message requires, as described herein. This routing request is preferably configured at the backend and is determined according to the contact details and/or preferences of the recipient.

At 1804, the message delivery status is optionally received from the carrier, if available. The message transaction is preferably captured in a chat history of communication between that authenticated user and the specific recipient at 1805. At 1806, all the transaction logs are synchronized to the customer's CRM in real time or in a batch process.

At 1807, the recipient receives the message in their default messaging app for that type of communication, such as an SMS message app on a mobile communication device for example. Preferably the default is set in advance and is stored in the previously described CRM. At 1808, the recipient receives the message with the code. The recipient then scans the code. At 1809, the contact is asked ask to navigate to its native messaging application, which then prepopulates the SMS template and the phone number as previously described.

This response request is then routed through the carrier at 1810. At 1811, the sender receives a notification of the response to the request, for example at their telephone number.

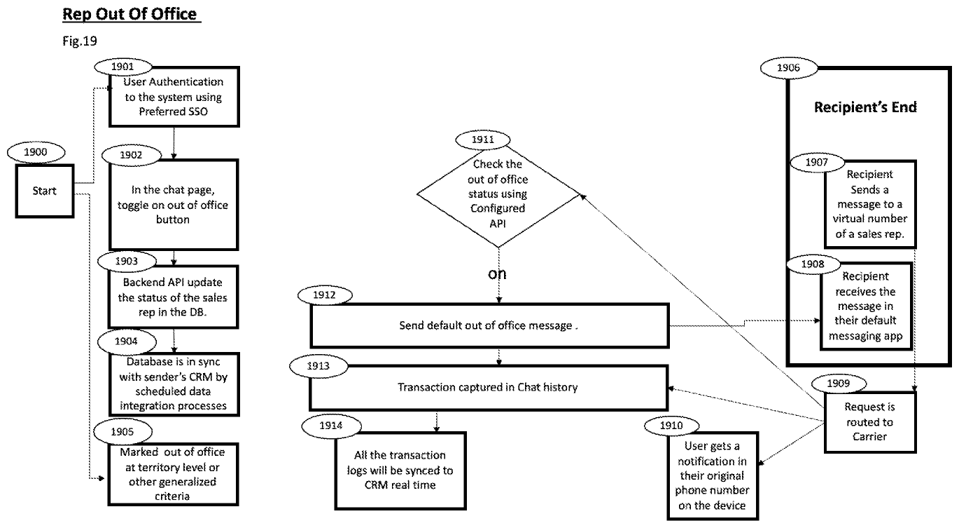

FIG. 19 shows an exemplary, non-limiting process for handling an out of office message. As shown, the user (who is the sender) may start the process by activating the system at 1900. The user is assumed for this example to be a sales representative or other representative of an external organization who wishes to contact a HCP or HCP staff, although other types of contacts may be included or substituted.

At 1901, the user performs user authentication to the system, for example using SSO (single sign on) technology as described herein. At 1902, optionally on the chat page, the user toggles on the out of office function. Alternatively a calendar connection may be used to determine the status of the user automatically. At 1903, the backend API updates the status of the sales representative in the system database. At 1904, the database is synchronized to the backend in real time or in a batch process. At 1905, the user is optionally marked out of office at the territory level or according to other generalized criteria.

Turning now to the recipient's side 1906, at 1907 the recipient sends a message to the external representative contact, such as the sales representative. The message may be sent to a virtual number. At 1908 the recipient receives the message in their default messaging app. At 1909, the request is routed to the suitable carrier as described herein. At 1910 the recipient then receives a notification on their own device, for example as a message. At 1911, the system checks the availability status of the recipient through the backend API to the database. As the out of office status is activated, at 1912, an out of office message is sent.

The message transaction is preferably captured in a chat history of communication between that authenticated user and the specific recipient at 1913. At 1914, all the transaction logs are synchronized to the customer's CRM in real time or in a batch process.

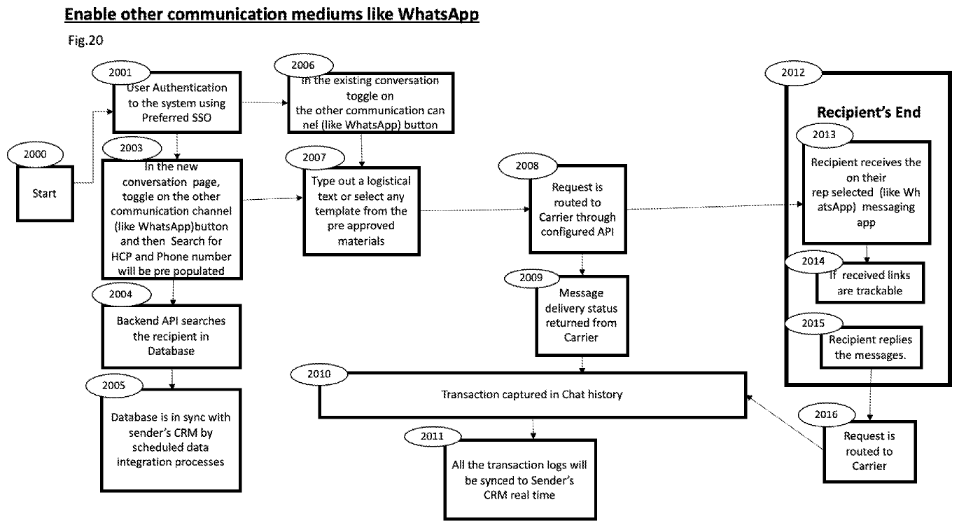

FIG. 20 shows a non-limiting, exemplary method for communication between an external support staff member and a recipient, who could be a healthcare professional (HCP) or HCP staff for example, using a specific example of a messaging service. This example for this method is the WhatsApp messaging service.

In this non-limiting example, the conversation is assumed to be between an HCP and an external support staff member. Preferably, this method is employed after communication is established between the healthcare professional and the external support staff member, whether generally or for this specific communication modality.

As shown, a healthcare provider, office staff member, external support staff member or other user initiates communication at 2000, for example by logging into a software interface. For the purpose of description only, the user is assumed to be an external support staff member attempting to contact an HCP (healthcare professional). The user in this non-limiting example is the sender of a message and the HCP is the recipient. The user is then invited to authenticate at 2001, for example by using an SSO (single sign-on) method, which may be performed through an existing email, social media or other user account. At 2006, the user (sender) may select an existing conversation to continue as described herein, after which the process continues at 2007

Alternatively, at 2003, the user may be shown a landing page and/or may employ a voice command to indicate which healthcare professional is to be contacted. If the phone number or other identifier of that healthcare professional is not available, then preferably the user enters it. Although the phone number is a preferred identifier for the healthcare professional, optionally a social media account identifier or other identifier may be used. Where suitable, such an identifier may be substituted for “phone number” when the latter is discussed herein. The user indicates that the messaging service to be used is WhatsApp. Alternatively, this messaging service selection may be made automatically according to the recipient's contact preferences.

At 2004, the backend API receives the request and searches for the correct healthcare provider information, for example in the previously described server database. The healthcare provider information may include for example contact details of the healthcare provider to be contacted, including but not limited to phone number and/or social media contact information. Optionally such contact information is for a “cut-out” communication modality, in which the private contact details of the HCP are kept private, while contact information that the server supports is provided. At 2005, the database is preferably synchronized with the CRM (customer relationship management) database of the external support staff company or organization by scheduled data integration processes. For example, such an synchronization may be performed through the client data integration API of FIG. 1 (not shown).

At 2007, the external support staff member determines the message structure and content, for example from one or more templates, and/or as free-form text, or a combination thereof. For example and without limitation if the healthcare professional has previously requested assistance with a particular good or service, and/or is requesting information, then a suitable template may be selected, for example to request that the healthcare professional call the external support staff member, login to a secure portal, or otherwise perform some action. Alternatively or additionally, the external support staff member may transmit information directly in the message. Optionally the external support staff member may request the HCP to perform some other action such as a booking an appointment. The text may also comprise logistical text as described herein.

At 2008, the request to send the message is routed as necessary to transmit it as previously described. In this non-limiting example, the message is a WhatsApp message and the request is routed to the WhatsApp service provider.

At 2009, the delivery status is returned from the message communication provider, which in this non-limiting example is the WhatsApp service, if available. The message transaction is preferably captured in a chat history of communication between that authenticated user and the specific recipient at 2010.

At 2011, all the transaction logs are synchronized to the customer's CRM in real time or in a batch process as previously described.

At 2013, the recipient receives the message in their default messaging app for that type of communication, which in this non-limiting example is the WhatsApp message service. In this non-limiting example, the recipient is assumed to be a healthcare professional. Again, preferably the default is set in advance and is stored in the previously described CRM. At 2014, any received links that have been sent through the message may be tracked; alternatively or additionally, any clicked links may be tracked.

At 2015 the recipient replies, whether because the message content requested a reply or independently. At 2016, the request is routed through the carrier for a WhatsApp message.

FIG. 21 shows a non-limiting, exemplary architecture for content moderation. The architecture receives user input at 2100, which is then routed to the content moderator module 2101. At the top branch, the raw input is preferably sent to a sentence modifier 2102, for example to check spelling of the words and/or grammar, and to modify or correct if necessary. A NLP (natural language processing) module preferably extracts meaning of the words and also of the sentences at 2103. A plurality of different processes may be applied to extract such meaning, shown as a wordnet 2104, to determine the meaning of individual words and/or their similarity to one or more stop words; and a sentence NLP analyzer 2105. Sentence NLP analyzer 2105 may apply various analysis techniques, including without limitation the BERT, ROBERTA and/or SENTENCEBERT techniques. These techniques may analyze word meaning in context and/or whole sentences or phrases. The output of both meaning analysis modules is then preferably fed to a similarity score module 2106, to determine similarity with one or more stop words, and/or other types of prohibited and/or controlled content. The latter may relate to phrases or sentences with permitted words but prohibited and/or controlled overall meaning. If the content requires further regulation, then the content may be passed to a further moderation process 2107. Alternatively, it is preferably passed to a process that does not require further moderation at 2108.

The lower branch relates to a process in which a plurality of predetermined stop words are analyzed to find similar words, and both the original words and these similar words may be further compared to the raw content. This process preferably relies on word comparison. As a non-limiting example, three stop words, stop words 1, 2 and 3, are received through processes 2109, 2110 and 2111, respectively.

Next, stop words 1-3 are analyzed to find similar words, for example by checking through a dictionary and/or by using stemming or other techniques. These similar words are then compared to the raw content, for example by using regular expressions at processes 2112, 2113 or 2114 for stop words 1, 2 or 3, respectively. Any detected stop words preferably cause the content to be sent to a further moderation process 2115. Alternatively, if no stop words are detected, then the content is preferably passed to a process that does not require further moderation at 2116.

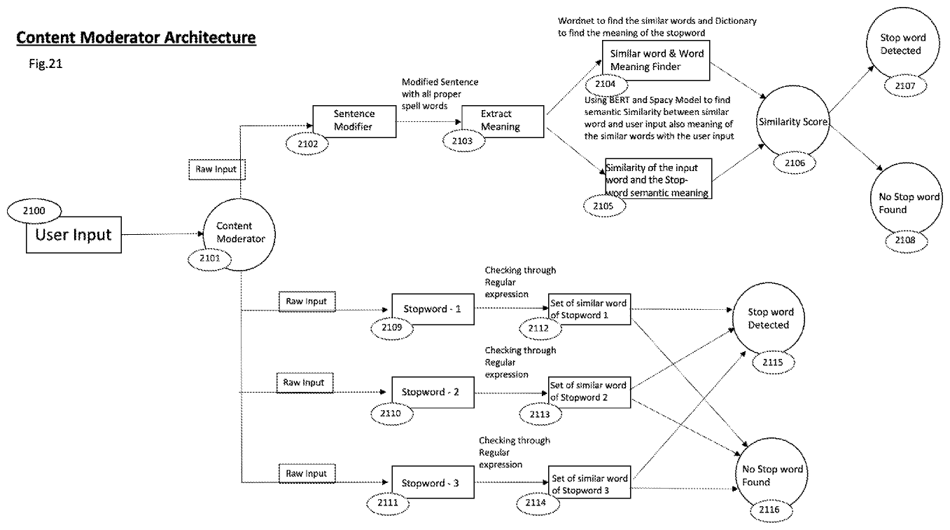

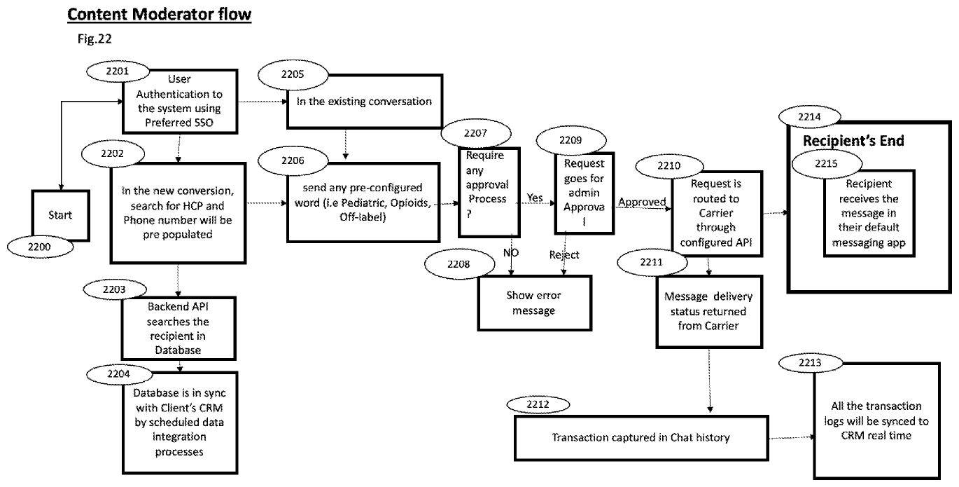

FIG. 22 relates to a non-limiting, exemplary method for content moderation of transmitted messages through the system as described herein. Steps 2200-2205 may be performed in a similar manner to steps 900-905 of FIG. 9.

At 2206, the user (sender) enters one or more words of the message, and/or selects a pre-created message, and/or edits a pre-created or suggested message. The suggested message may be generated by an AI model. The words are then analyzed for content moderation purposes, for example as described with regard to FIG. 21. For example, the content may be analyzed to detect one or more stop words, relating to restricted, controlled and/or prohibited content. The extent to which content requires moderation may be determined by organization policies. For example, for a distributor or manufacturer of pharmaceuticals, such content may relate to controlled substances, such as opioids for example; off-label use of a pharmaceutical (that is, a use that is not strictly allowed by a drug regulation agency); or pediatric uses.

At 2207, if the content does not require moderation, then it may be passed directly to 2210, for routing through the carrier as previously described. If the content does require moderation, but no moderation is available, then an error is raised at 2208 and the process preferably stops. If moderation is available, then at 2209, the content is reviewed. If allowed, then it is passed to the carrier at 2210.

From the carrier, the message delivery status is preferably returned at 2211. The message transaction is preferably captured in a chat history of communication between that authenticated user and the specific recipient at 2212.

At 2213, all the transaction logs are synchronized to the customer's CRM in real time or in a batch process as previously described.

At 2214, the recipient receives the message. The message is preferably received at 2215 in their default messaging app for that type of communication, which in this non-limiting example is the WhatsApp message service. In this non-limiting example, the recipient is assumed to be a healthcare professional. Again, preferably the default is set in advance and is stored in the previously described CRM. At 2014, any received links that have been sent through the message may be tracked; alternatively or additionally, any clicked links may be tracked.

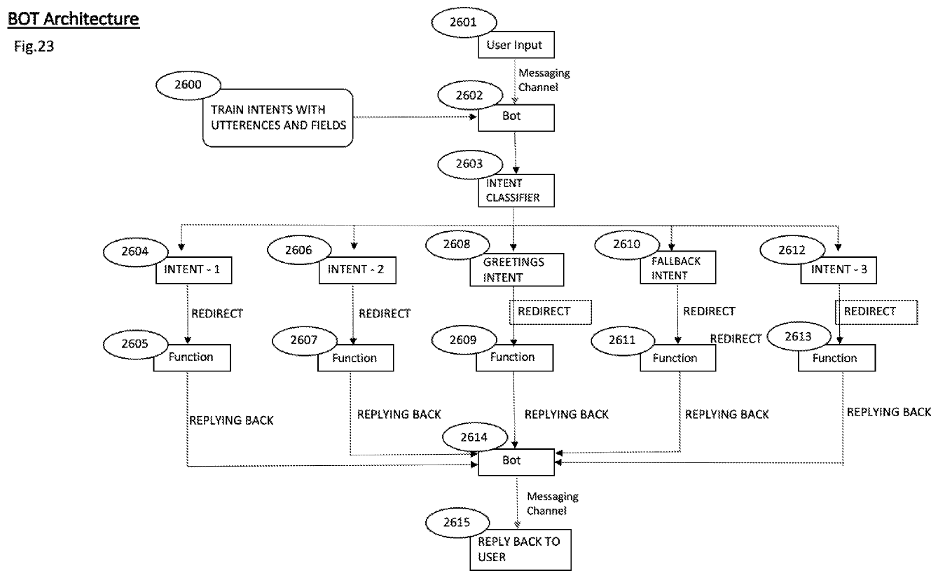

FIG. 23 relates to a non-limiting, exemplary architecture for bot communication. The architecture preferably trains the bot with a training module 2600. Training module 2600 preferably trains the bot on a combination of text words, sentences and phrases, and also previously determined intents. The intents may relate to a predicted intention of a sender of a message, including but not limited to, requesting information, registering a complaint, asking for a status update on a process, and/or information and/or a physical product that is expected to be received, and so forth.

The bot 2602 is trained by training module 2600, and then receives a user (or other sender) input 2601. Bot 2602 then classifies the intent of the input through an intent classifier 2603. Certain types of input may relate to a generic intent, including but not limited to a greetings intent 2608. If this intent is detected, then the input is preferably redirected to a further analysis function 2609, to see if any additional information is contained in the input. The intent plus any further information is then preferably passed to a response bot 2614. Response bot 2614 preferably is capable of NLG (natural language generation). Response bot 2614 then sends a response back to the user (sender) at 2615, according to the intent and the further information, if available.

Other intents may relate to specific types of communication, for example as previously described. These intents may be separately detected and are shown as intents 1, 2 and 3 for the purpose of illustration only and without any intention of being limiting. Intents 1-3 are received at 2604, 2606 or 2612 as shown, respectively. The analysis then performed as described with regard to further analysis function 2609 for the greeting intent, to see if any additional information is contained in the input, at functions 2605, 2607 or 2613, respectively. The process then continues as previously described.

If the input is not understood, or if no suitable intent analysis function is available, then the intent may be determined to be a fallback intent at 2610. Further information related to the fallback may be determined at function 2611. The process then continues as previously described.

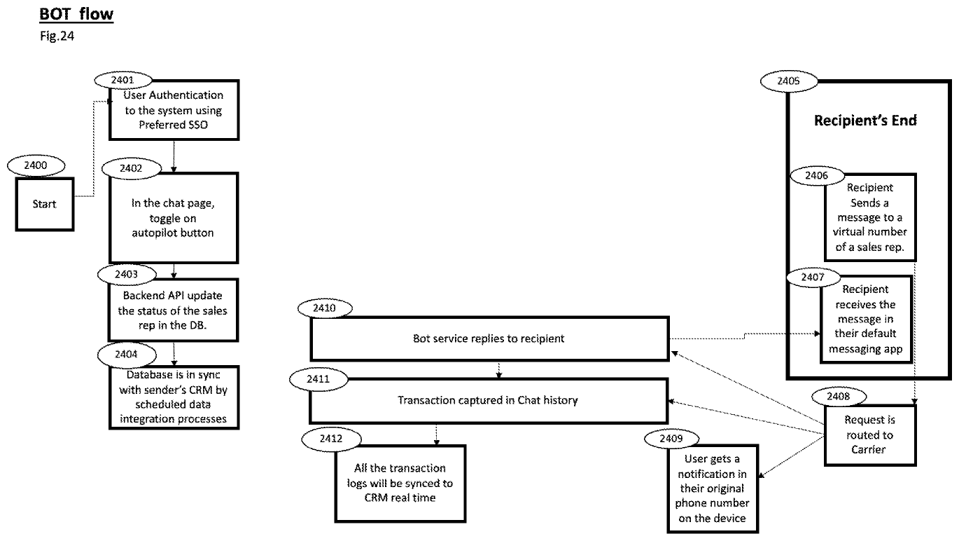

FIG. 24 relates to a non-limiting, exemplary method for bot communication. . As shown, the user (who activates the bot) may start the process by activating the system at 2400. The user is assumed for this example to be a sales representative or other representative of an external organization who wishes to contact a HCP or HCP staff, although other types of contacts may be included or substituted.

At 2401, the user performs user authentication to the system, for example using SSO (single sign on) technology as described herein. At 2402, optionally on the chat page, the user toggles on the automatic reply (bot) function, so that the bot is able to respond in place of the user for example. Alternatively the bot function may be triggered automatically, for example when an out of office status is set. At 2403, the backend API updates the status of the sales representative in the system database, in terms of the activation of bot communication. At 2404, the database is synchronized to the backend in real time or in a batch process.

Turning now to the recipient side at 2405, the recipient may initiate a message communication session by sending a message at 2406. Alternatively, the message communication session may be initiated at the recipient side by receiving a message at 2407. In either case, message sending and receiving then continues as described herein. When the recipient sends a message at 2406, for example through their default messaging app, the message is preferably routed to the carrier at 2408 as previously described.

At 2409, the user may optionally receive a notification on their own device, for example as a message. At 2410, the bot service engages with the recipient to send and receive messages. The sent message is then routed back to the recipient, preferably at the default messaging app as previously described.

The message transaction is preferably captured in a chat history of communication between that authenticated user and the specific recipient at 2411. At 2412, all the transaction logs are synchronized to the customer's CRM in real time or in a batch process.

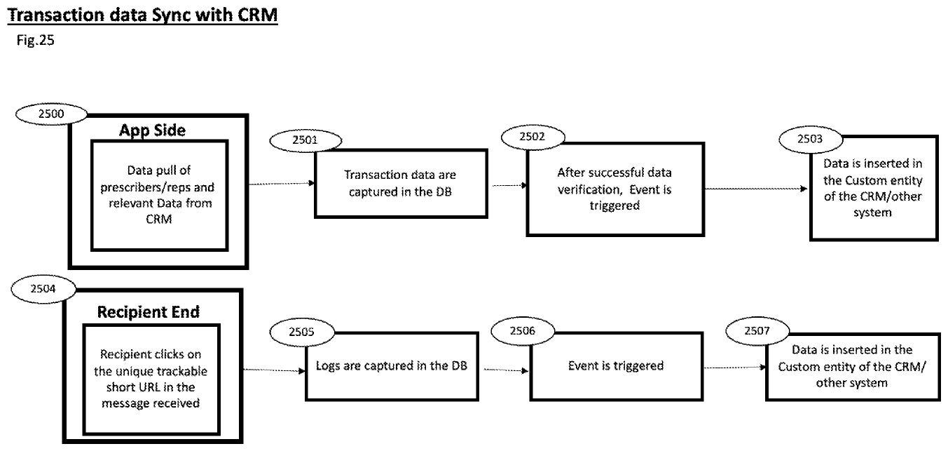

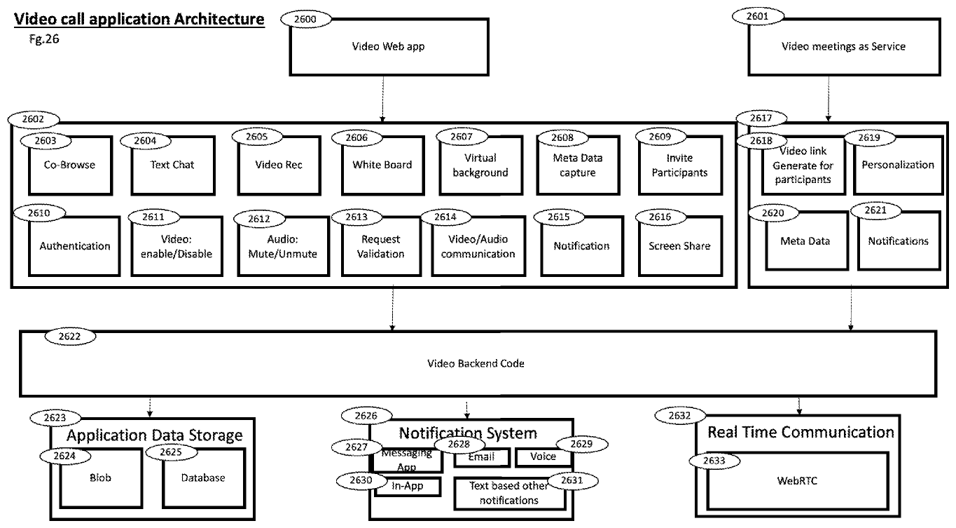

FIG. 25 shows a non-limiting, exemplary method for communication logging into a database. The method synchronizes information taken from communication records with the database. The database may for example be used by an external support staff member to coordinate customer information, in which the HCP and/or the HCP staff member may be a customer of the company employed by the external support staff member. Turning now to FIG. 25, at 2500, transaction information is captured from the message application of the HCP and/or HCP staff member, such that incoming/outgoing messages or calls from the application, and/or associated metadata, may be captured. At 2501, logs of such captured information are stored in the database, such as a CRM for example. At 2502, an event is triggered, for example according to the identity of the HCP and/or HCP staff member, and/or of the external support staff member. At 2503, data is inserted in the custom entity of the CRM, according to the triggered event, for example to update particular information.

On the recipient side, at 2504, with the recipient in this non-limiting example being the HCP and/or HCP staff member, the process preferably begins when the recipient clicks on the unique trackable short URL in the message received. At 2505, a log of such a click and any associated information, such as the message contents and/or the associated web page or micropage contents, are stored in the database, such as a CRM for example. At 2506, an event is triggered, for example according to the click of the URL and/or according to the identity of the HCP and/or HCP staff member, and/or of the external support staff member. At 2507, data is inserted in the custom entity of the CRM, according to the triggered event, for example to update particular information.

FIG. 26 relates to a non-limiting, exemplary architecture for a video call. The video call architecture may be supported through a video web app 2600. As shown, video web app 2600 features a plurality of components 2602, including but not limited to a co-browsing module 2603, which enables participants to view and optionally also interact with a web page together. For co-browsing module 2603, as well as for other modules, optionally only one or some participants may use or activate such a module.

A text chat module 2604 may be present to enable participants in the video session to communicate by chat during the session. A video recording module 2605 may supporting capture of the data of the video session, including without limitation, recording audio and video, and optionally also capturing chat. A white board module 2606 may be provided to enable participants to draw on a white board during the video session. A virtual background module 2607 may enable video participants to change the background of their video capture.

A capture meta data module 2608 may also capture meta data relating to the computational device being used to view the video session, and/or in relation to the time of day, date, day of the week and so forth. Such meta data may be captured for security reasons and/or to be included with a recording of the video session.

An invitation module 2609 may enable participants to further invite additional participants to the video session, or to remind invited participants who have not yet joined to enter the video session.

An authentication module 2610 is preferably used to authenticate participants in the video session. Such authentication may be for limited participation, with limited access to one or more modules as described herein; or may permit additional access to such one or more modules. Preferably all participants engage with authentication module 2610 before entering the video session.

A video control module 2611 preferably enables each participant in the video session to determine whether to show their video. Alternatively, video from one or more participants may be blocked by video control module 2611, for example according to access privileges as described herein. An audio control module 2612 may perform a similar function for audio.

A request validation module 2613 may validate the format of a video session request, for example to determine whether the required parameters are available.

A communication module 2614 may support video and/or audio communication between the participants.

Optionally, a notifications module 2615 notifies a requester of the signature, the signing entity or both, or other entities, upon signing of the form or other content. Notifications and other data may be passed to a video backend 2622.

A screenshare module 2616 may enable one or more participants to share their computer screens with other participants.

The video call architecture may also be supported through a video meetings service 2601. Such a service may be operated through a plurality of components 2617 as shown, including without limitation an unique URL generator 2618, which may create a unique URL for this video session, whether for all participants or for a single participant (in which case, preferably each participant receives a unique URL). A personalization module 2619, a meta data capture module 2620 and a notification module 2621 may operate as previously described.

Video backend 2622 then preferably sends the transmitted information, which may include one or more of verification of the signature, the form information or other content, meta data, any security verification such as IP address verification, and so forth, to an application data storage 2623. Such information may then be stored in a blob form 2624 and/or a database 2625.

A notification system 2626 optionally receives notifications from the previously described notification modules, and then sends a notification through a suitable messaging modality, including but not limited to a message service 2627, an email service 2628, a voice service 2629, an in-app service 2630 and/or another type of text based notification 2631.

An real time communication service 2632 may feature access to other services, including but not limited to a WebRTC service 2633.

Optionally, the video call architecture may be provided as a video feature, for example as a widget, such that an option to start a video call may be embedded at different locations. For example, such a widget may be embedded on a web page or in another app.

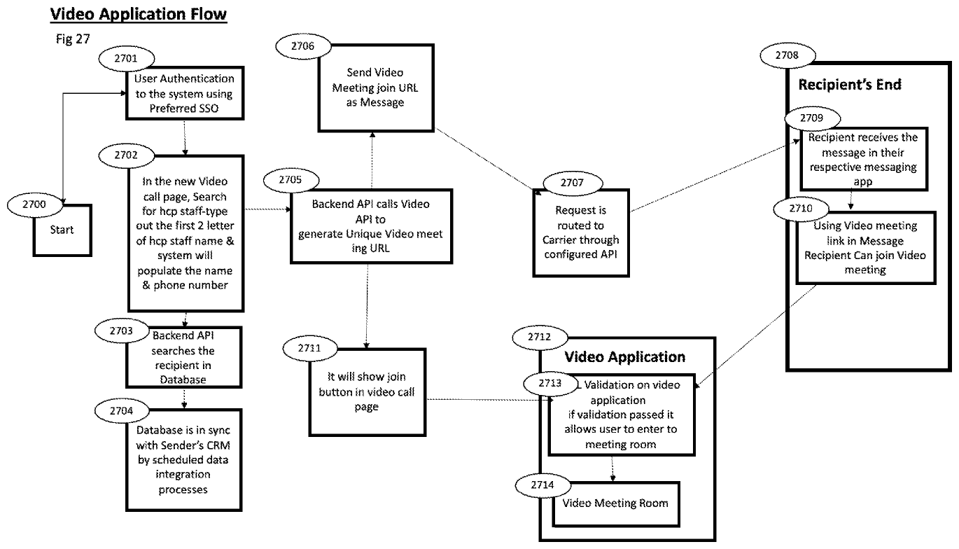

FIG. 27 relates to a non-limiting, exemplary method for a video call. As shown, stages 2700-2704 may be performed in a similar manner as stages 900-904 of FIG. 9. At 2705, the backend API preferably calls the video system API to generate at least one unique video session URL; optionally one such URL is generated per participant. At 2706, the video invitation URL is sent to at least one participant as a message. At 2707, the requested is routed through the carrier as previously described. At the recipient 2708, the recipient receives the message with the video link in their respective messaging modality at 2709. At 2710, the recipient joins the video meeting by clicking on the video link and/or by entering meeting information into a video meeting app or webapp.

Turning back to 2711, the backend preferably sends validation and/or authentication information, and optionally other details about the meeting (including for example permission(s) granted to the participants) to video application server 2712. At 2713, according to such validation information, server 2712 determines whether the recipient is permitted to enter the meeting room. If validated, then the recipient enters at 2714.

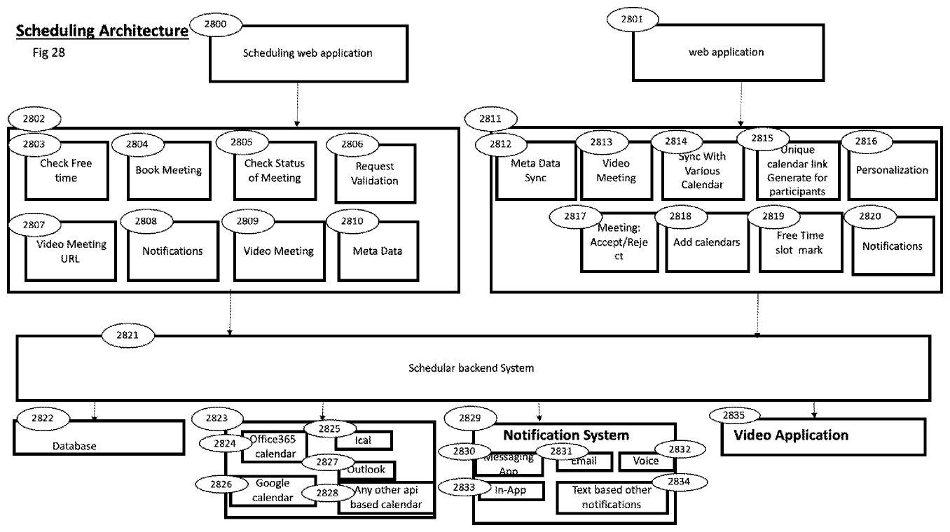

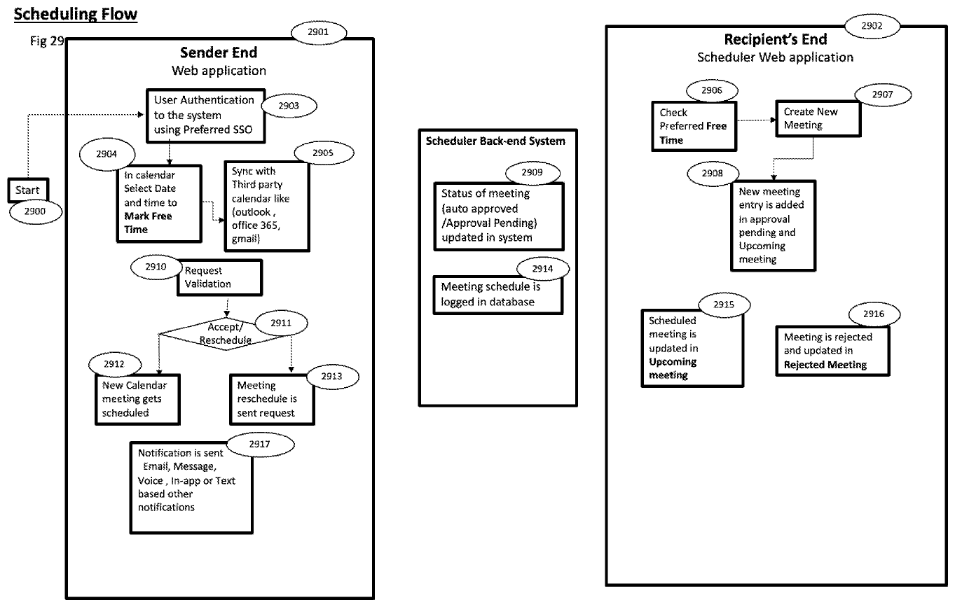

FIG. 28 relates to a non-limiting, exemplary architecture for scheduling a call. The scheduling architecture may be supported through a scheduling web app 2800. As shown, scheduling web app 2800 features a plurality of components 2802, including but not limited to an availability searching module 2803, which supports finding an available time for at least one and preferably a plurality of participants.

A meeting booking module 2804 enables the video meeting to be scheduled, either after receiving an available time for one or more participants, or according to a manual scheduled meeting. The status of the meeting may be checked at 2805, for example to determine whether one or more participants have indicated that they cannot attend the meeting. A validation module 2806 is preferably used to validate participants who are to be scheduled in the meeting. Such validation may be for limited participation, with limited access to one or more modules as described herein; or may permit additional access to such one or more modules.

A video meeting URL generator 2807 may create a unique URL for this video session, whether for all participants or for a single participant (in which case, preferably each participant receives a unique URL).

Optionally, a notifications module 2808 notifies such information as acceptance of a meeting, change in status for a participant, change in a meeting schedule and so forth. Notifications and other data may be passed to a scheduling backend 2821.

A video meeting module 2809 may support scheduling and/or setup of a video meeting as described herein. A capture meta data module 2810 may also capture meta data relating to the computational device being used to view the video session, and/or in relation to the time of day, date, day of the week and so forth. Such meta data may be captured for security reasons and/or to be included with a recording of the video session.

The scheduling architecture may also be supported through a scheduling service 2801. Such a service may be operated through a plurality of components 2811 as shown, including without limitation a meta data synchronization module 2812. Meta data synchronization module 2812 preferably collects meta data for the purpose of analytics, for example to determine who is scheduling a meeting and with whom, and also in regard to meeting completion rate, and so forth. A video meeting module 2813 may support creation of and/or the provision of a video meeting as described herein. A calendar synchronization module 2814 supports synchronization of a plurality of participant calendars, for example according to time zone and/or to send a meeting invitation. A calendar link generator 2815 optionally generates a unique calendar link for the scheduled meetings, optionally uniquely for each participant but preferably at least unique to the scheduled meeting. A personalization module 2816 may optionally personalize calendar choices, the preferred days and/or times for a meeting, and/or the invitation text, or other items. A meeting accept or reject module 2817 enables each participant to accept or reject a meeting, and optionally to request rescheduling.

A calendar addition module 2818 optionally enables a participant to share their calendar with the scheduling system, for example for automatic scheduling and/or to indicate preferred days and/or times for a meeting. A free time slot module 2819 may be used to positively indicate preferred and/or open meeting times for each participant. A notification module 2820 may notify such information as acceptance of a meeting, change in status for a participant, change in a meeting schedule and so forth. Notifications and other data may be passed to a scheduling backend 2821.

Scheduling backend 2821 preferably receives information about the scheduled meeting, the participants and optionally other details, and then preferably sends the transmitted information, which may include participant identification information, meta data, any security verification such as IP address verification or validation, and so forth, to a database 2822.

A calendar interface service 2823 optionally supports sharing and/or synchronization of, and adding of meetings to, one or more API based calendars as described herein. As shown, calendar interface service 2823 may include connections to calendars for Office 365 (2824); iCal (2825); Google calendar (2826); or any general API based calendar (2827).

A notification system 2829 optionally receives notifications from the previously described notification modules, and then sends a notification through a suitable messaging modality, including but not limited to a message service 2830, an email service 2831, a voice service 2832, an in-app service 2833 and/or another type of text based notification 2834.

A video meeting service 2835 may support video meetings as described herein, and may interact with the previously described scheduling web app 2800 and/or scheduling service 2801 through scheduling backend 2821.

FIG. 29 relates to a non-limiting, exemplary flow for scheduling a meeting as described herein, such as a video call. The flow is from a sender web application 2901 to a recipient web application 2902, through a process beginning at 2900. The flow is enabled by a scheduling backend, which may be implemented as described for example with regard to FIG. 28.

The flow is initiated at sender web application 2901, by user authentication to the system, for example using SSO (single sign on) technology, at 2903. The sender web application may then login to a calendar function to mark free and/or available time at 2904, and/or meeting preferences; or such information may be indicated separately. A specific requested meeting time may also be included. At 2905, the sender's calendar is preferably synchronized with the scheduling backend as previously described.

At 2906, at recipient web application 2902, preferred free time and/or preferred meeting times are checked. At 2907, a new meeting is created. At 2908, a meeting validation request is sent to the scheduling backend.

At 2909, the scheduling backend receives calendar information from sender web application 2901 and from recipient web application 2902. The status of the meeting is then updated in the system, such as for example pending approval, auto approved and so forth. At 2910, the sender web application 2901 requests validation to the scheduling backend. At 2911, the set meeting time is either accepted or rescheduled. If accepted, the meeting is then scheduled on the calendar at 2912. If the meeting time is to be rescheduled, then a reschedule request is sent at 2913 to the scheduling backend. The reschedule request is then logged at 2914 in the database of the scheduling backend.

At 2915, the scheduled meeting is updated, for example due to the previously described rescheduling request, and is accepted. Alternatively, the meeting time may be rejected at 2916.

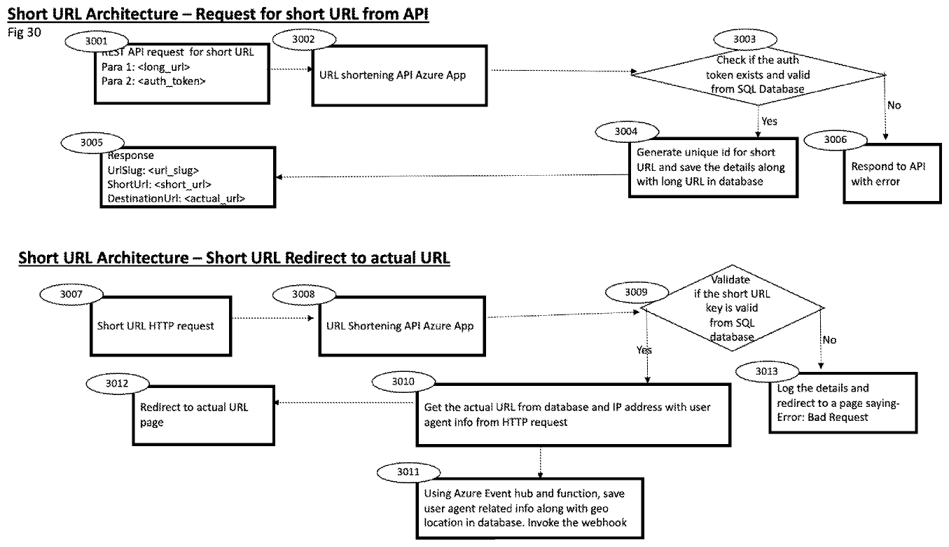

FIG. 30 relates to a non-limiting, exemplary architecture for short URL creation and redirection. The process of requesting the short URL starts at 3001, with a REST API request for a short URL. Preferably two parameters are passed: a long URL (that is to be shortened) and an authentication token. At 3002, these parameters are sent to a URL shortening app, such as the Azure URL shortening service. At 3003, the service determines whether the authentication token is valid. If so, then at 3004, a unique identifier and unique short URL are preferably created, and stored in the database, for correspondence to the long URL. At 3005, the response is sent, preferably with the short URL, and optionally with the long URL and a URL slug.

If the token is not valid, then at 3006, an error is sent in response to the API request.

The process of redirecting the short URL to the long (initial) URL starts at 3007 with a redirect request. At 3008, the application that created the short URL preferably receives it and starts the redirection process. At 3009, the short URL is validated, for example by checking against the database. At 30010 if validation succeeds, then the actual URL is retrieved from the database. Optionally, the IP address with the request information from the HTTP request is also stored, for example for security or logging purposes, at 3011. At 3012, the expanded URL is returned to the requester.

If the short URL is not validated, preferably an error message is returned at 3013.

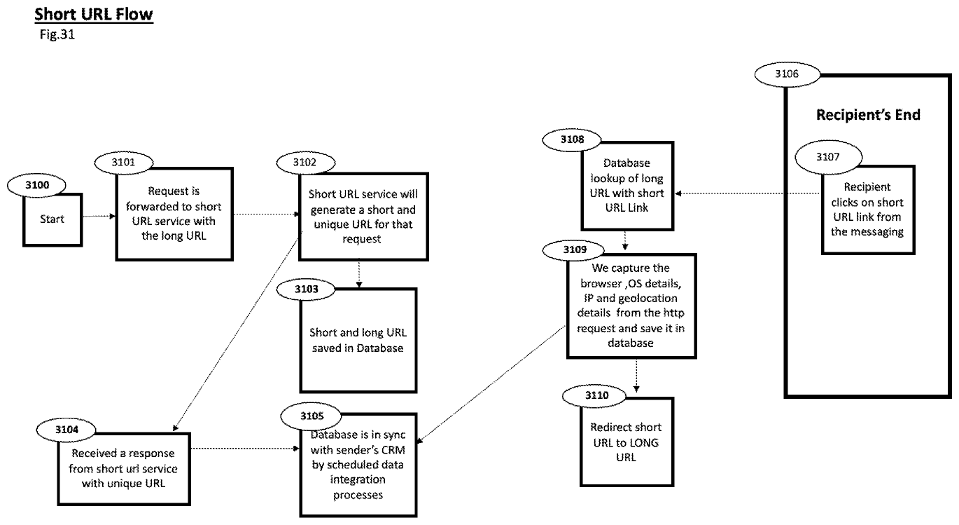

FIG. 31 relates to a non-limiting, exemplary flow for short URL creation and redirection. As shown, the process starts at 3100, when a request is sent to the short URL service, including a long URL that is to be shortened, at 3101. At 3102, the short URL service generates a short and unique URL for that request, which is then stored in the database at 3103. At 3104, the unique short URL is sent in response to the request. At 3105, the database is synchronized with the CRM of the user, to include both the short URL and the original long URL.

At the recipient side 3103, the recipient receives the message. At 3107, the recipient clicks on the short URL link from the messaging. At 3108, the short URL redirects to the correct longer URL, through a database lookup. At 3109, various meta details are preferably obtained, including but not limited to the browser, operating system details, IP and geolocation details from the http request. These details are then saved in a database. At 3110, the redirect request itself is performed.

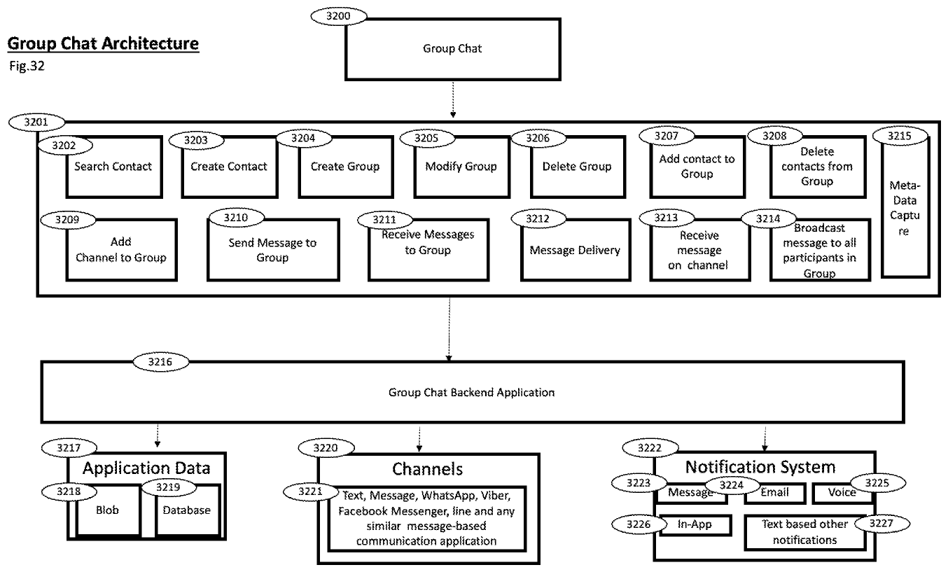

FIG. 32 relates to a non-limiting, exemplary architecture for a group chat. The group chat system 3200 preferably features a plurality of components 3201. These components may include but are not limited to searching a contact (3202), creating a contact (3203), creating a group (3204), modifying a group (3205), deleting a group (3206), adding a contact to a group (3207), deleting contacts from the group (3208), adding channel to the group (3209), sending a message to the group (3210), receiving a message by the group (3211), sorting message delivery (3212), receiving a message on channel (3213), broadcasting a message to all participants in a group (3214), and/or meta data capture (3215).

The group chat system 3200 then preferably communicates with a Group Chat Backend 3216. Group Chat Backend 3216 then preferably sends the transmitted information, which may include one or more of chat information, group addresses, meta data, any security verification such as IP address verification, and so forth, to an application data storage 3217. Such information may then be stored in a blob form 3218 and/or a database 3219.

Channels are preferably handled through a channels module 3220, which is preferably able to handle a plurality of channels, for example those selected from the group consisting of SMS messages, WhatsApp, Viber, Facebook Messenger, Line, any suitable similar message-based communication applications, and so forth.

A notification system 3222 optionally receives notifications from the previously described notification modules, and then sends a notification through a suitable messaging modality, including but not limited to a message service 3223, an email service 3224, a voice service 3225, an in-app service 3226 and/or another type of text based notification 3227. Notification system 3222 may for example send messages to the group chat through one or more different messaging systems. Optionally messages are routed through a plurality of messaging systems.

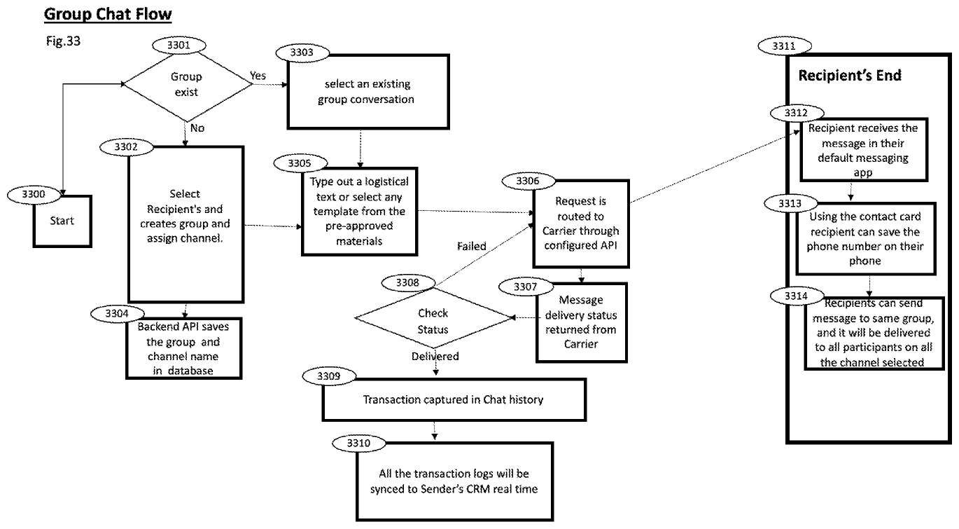

FIG. 33 relates to a non-limiting, exemplary flow for a group chat, between any suitable combination of a HCP, a staff member of an office of a healthcare professional, a patient or an external support staff member. In this non-limiting example, the communication is between a HCP and/or the staff member of the healthcare professional, and the external support staff member. The external support staff member initiates communication at 3300, for example by logging into a software interface, and then by authenticating, as previously described.

At 3301, the user determines whether the group exists. If not, then at 3302, the user selects the recipients (other group participants) and creates the channel. As noted below, a plurality of different messaging modalities may be used for the recipients, and/or a different messaging modality may be used for the user creating the group than for one or more other recipients. At 3304, the backend API saves the group details and channel name, or other identifier, in the database.

At 3303, if the group does exist then the existing group conversation is selected. At 3305, the external support staff member determines the message structure and content, for example from one or more templates, and/or as free-form text, or a combination thereof. For example and without limitation if the external support staff member wishes to transmit information or alternatively wants the HCP staff member to contact them, then a suitable template may be selected, for example to request that the HCP staff member call the external support staff member, the HCP and/or the office; login to a secure portal, or otherwise perform some action. Alternatively or additionally, the external support staff member may send information directly in the message.

At 3306, the request to send the message is routed as necessary to transmit it as previously described. As a non-limiting example, the message is an SMS message and the request is routed to a suitable carrier. As another non-limiting example, the message is transmitted through a different messaging modality, which may for example comprise a social media channel modality, in which case the request is routed to another suitable carrier.

At 3307, the delivery status is returned from the carrier or other message communication provider. At 3308, the delivery status is checked. If the message delivery status is failed, then preferably stages 3306 and 3307 are repeated. If the message delivery status is successful, then the message transaction is preferably captured in a chat history of communication between that authenticated user and the specific recipient at 3309. At 3310, all the transaction logs are synchronized to the customer's CRM in real time or in a batch process as previously described.

Turning now to the recipient's side at 3311, the message is received in the messaging modality app of the recipient at 3312, according to the modality used to send the message. The modality selected may be a default for the recipient, for example. Optionally, the message features a contact card. At 3313, the recipient may save the phone number, or other social media messaging contact details, or other messaging modality contact details. Such details may be saved only for the user (external staff member) and/or for one or more other participants in the group chat. The recipient may save such information on a mobile device, such that 3311 may represent the mobile communication device, including but not limited to a cellular telephone, a smart phone and the like.

At 3314, each recipient may then send a message to the same group included in the group chat, such that it is delivered to all participants on the selected messaging modality for each participant.

Optionally, multiple messaging modalities may be used for communication by different members in the group for the group chat. In such a situation, stages 3306-3308 are performed for each messaging modality separately, while the system tracks message transmission, content and receipt for compliance and auditing purposes.

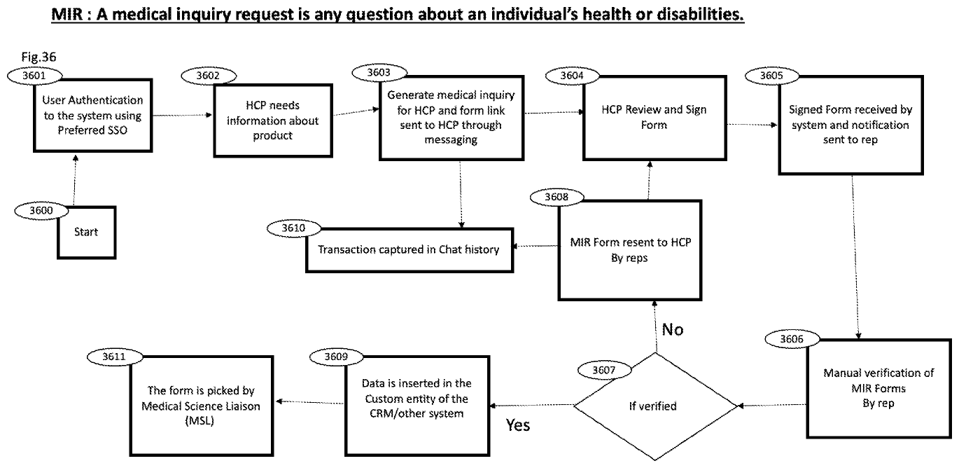

FIG. 36 relates to a non-limiting, exemplary method for handling a MIR request. The process begins at 3600, for example through a SSO login or other authentication process at 3601. At 3602, the user receives a request for information from a HCP contact. The request for information is in the form of a MIR request, which may be the result of the process described with regard to FIG. 16. Stages 3603-3605 may be performed according to the process as described with regard to FIG. 16.

At 3606, the filled in MIR request form is reviewed, whether manually by a sales representative or other agent, or through a NLP (natural language processing) algorithm. For example, the information in the MIR request form may be analyzed with regard to stop words and/or content that requires further regulation. At 3607, the result of the verification process may be reviewed to determine success or failure of verification. At 3608, if verification was not successful, the MIR form may be resent to the HCP. Otherwise, at 3609, if verification was successful, then data from the form may be inserted to a database and/or CRM, preferably associated with the HCP. The data may also be entered to a ticketing system, to be assigned to qualified personnel for reviewing the request. The data may then be pulled at 3611 by qualified personnel, such as for example a Medical Science Liaison (MSL). Also at 3610, each transaction in the system is preferably logged.

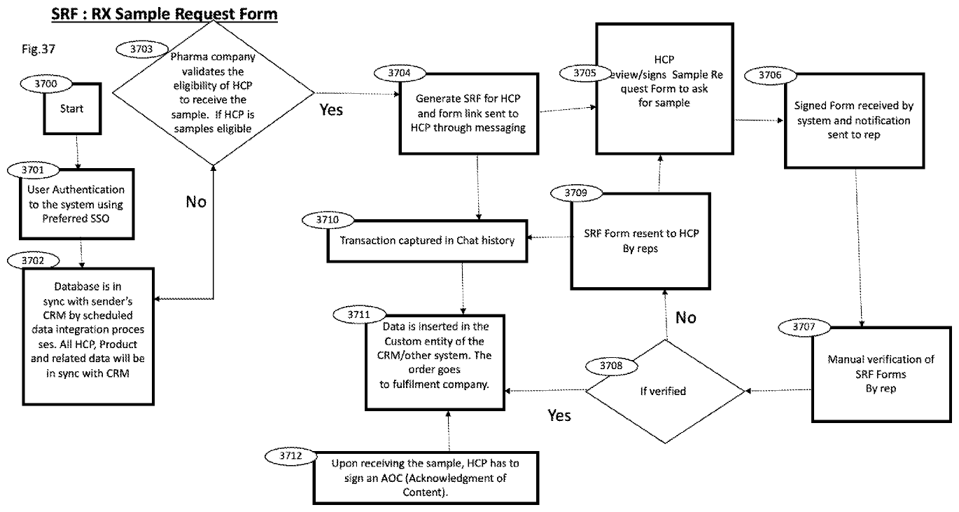

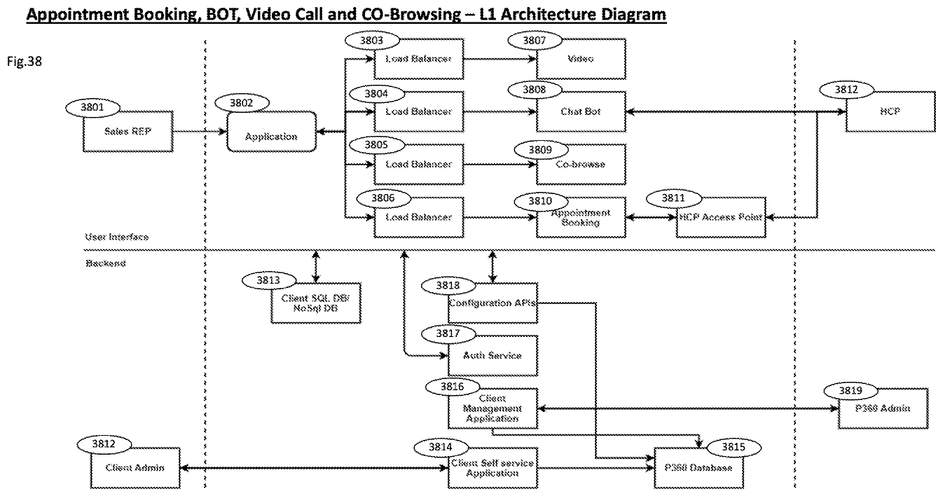

FIG. 37 relates to a non-limiting, exemplary method for handling a sample request, for a physical sample of a medical device, pharmaceutical, diagnostic kit or other medical product. The process starts at 3700, when the user authenticates to the system at 3701 as previously described. At 3702, the user reviews the database for the contact details of the HCP. Physical samples are expected to be more strictly regulated than information, such that preferably the process of fulfilling a request for a physical sample is not started unless the contact details of the HCP are already in the database and/or CRM.

At 3703, these contact details are sent to the company providing the sample, which is in this non-limiting example is a pharmaceutical company. The company preferably verifies that the HCP is eligible to receive the sample. For example, for some types of samples, licensing requirements may prohibit the company from sending the sample unless the HCP has that particular license. If the HCP is not eligible, then the process preferably stops.

At 3704, if the HCP is eligible, then a SRF (sample request form) is generated for the HCP and is sent with a signing link through a messaging modality. At 3705, the HCP reviews and signs the SRF as previously described with regard to the signing process herein. At 3706, the signed form is received and is sent to the sales representative or other agent of the company. At 3707, the SRF form may be reviewed as described in FIG. 36 with regard to the MIR form. At 3708, the verification result is reviewed, to determine whether it was successful or not.

If not successful, then at 3709, the SRF is generated and sent again to the HCP. The process then restarts. For example, the process may not be successful because the HCP has requested that the sample be sent to a medical office, clinic or other setting which does not fall under the license of the HCP or other licensing for that type of product. Such an additional level of control may be applied for controlled substances, such as opioid painkillers for example. Whether successful or not, all messaging transactions are preferably logged at 3710.

If the process at 3708 is successful, then at 3711, data regarding the SRF is preferably stored in a CRM or other database. A ticket may also be generated to enable fulfillment of the requested sample. Upon receiving the sample, the HCP signs an AOC (Acknowledgment of Content) at 3712, to acknowledge receipt of the sample. This acknowledgment is also preferably stored in the CRM or other database.

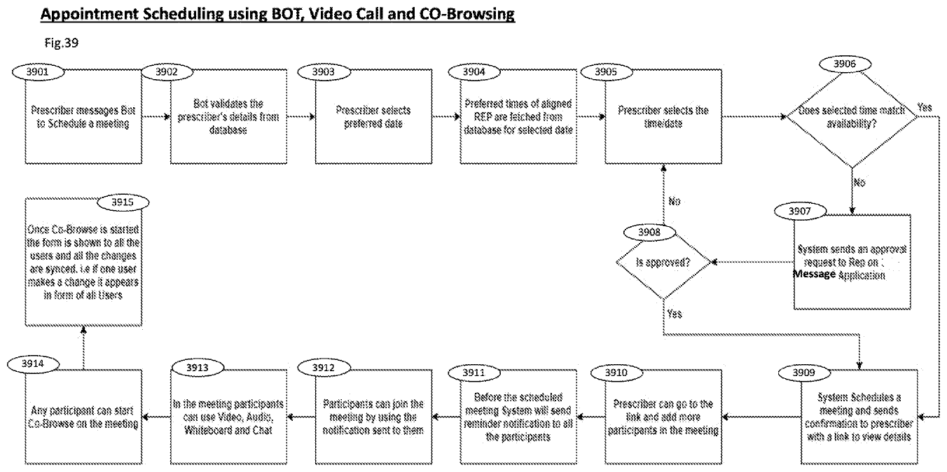

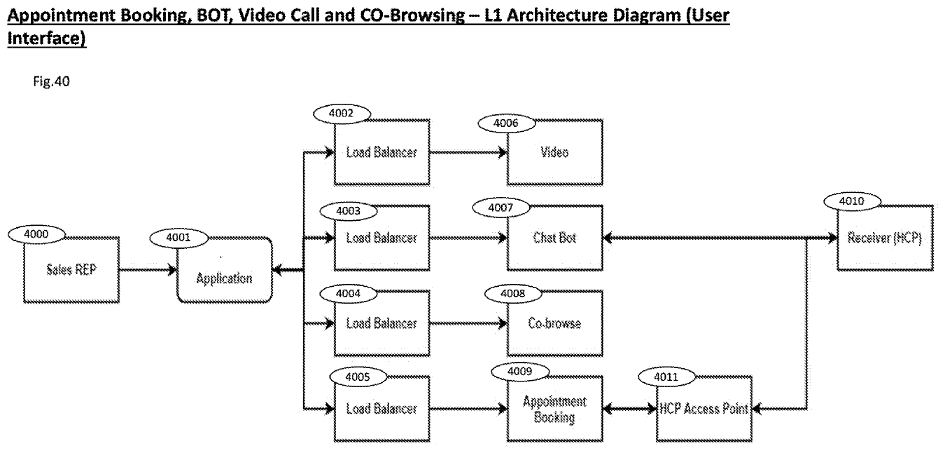

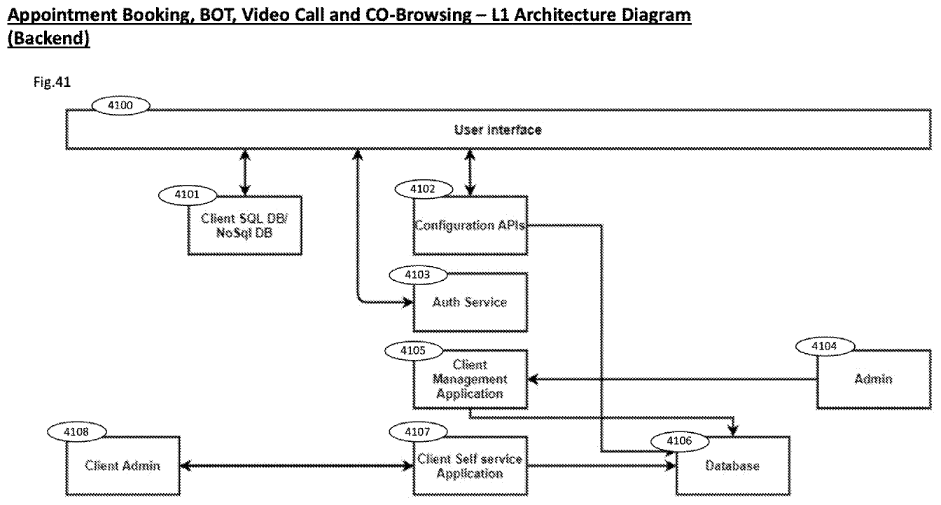

FIG. 38 shows a system for supporting communication with regard to multiple modalities, while FIG. 39 shows a non-limiting, exemplary method for communication through such a system. The operation of FIG. 38 is described with regard to FIGS. 40 and 41, which shows the system of FIG. 38 in more detail.

As shown, the process begins at 3901 when a HCP, described herein as a prescriber as a non-limiting example, messages a bot or other automatic communication software through a computational device and a messaging modality, to schedule a meeting. For example, the recipient may use SMS to message the bot. At 3902, the bot verifies the recipient's details through a database. Optionally only after verification is complete and a return message is received from the bot, or alternatively before such verification is complete, the recipient selects a preferred date at 3903. At 3904, the preferred times for a particular representative, medical professional, external support staff or another entity are shown. At 3905, the recipient selects the time and date for the appointment. At 3906, the system checks whether the selected time matches availability for the entity who will meet. If not, then at 3907, the system sends a message to that entity to see if in fact such a meeting is possible. The entity responds at 3908. If the meeting is not possible, then the process returns to 3905.

If a time and date are considered to be possible, either automatically through the system or alternatively through a manual approval process, then at 3909, the system schedules a meeting and sends the details to all participants, preferably including at least the recipient who initiated the meeting and the entity with whom the meeting was scheduled. For example, if the recipient who initiated the meeting is a medical professional, and the entity with whom the meeting was scheduled is a sales person or other representative of a company providing healthcare services and/or products, then preferably both the medical professional and the sales person or other representative receive the meeting details through the appropriate computational device. Optionally such meeting details include a link for the meeting and/or a calendar invite, and are sent through an appropriate message modality, which in this example may also include email.

At 3910, optionally the recipient is able to use the link to invite one or more additional participants to the meeting. At 3911, optionally a reminder is sent to all participants before the meeting is to start. At 3912, the participants are preferably able to join the meeting through the invitational link that was sent to them. At 3913, optionally multiple meeting modalities may be used, including but not limited to audio, video, whiteboard and/or chat. At 3914, optionally co-browsing is used to show online material to one or more meeting participants. Also optionally at 3915, co-browsing is shown to all participants and changes from one participant are synchronized to other meeting participants.

FIG. 40 shows a non-limiting, exemplary user interface system for communication through multiple modalities. In this system, a sender 4000, described as a sales representative in a non-limiting example, and a recipient 4010, described as a HCP as a non-limiting example, communicate with each other. Although the recipient is shown as the entity who is able to book an appointment, optionally the roles of sender and recipient may change, even for the same individual or entity, depending on the context. For example, in one situation, sender 4000 may be a healthcare professional, while recipient 4010 may be a patient, caregiver or an external support staff member for example. In another situation, sender 4000 may be a patient, caregiver or an external support staff member for example, while recipient 4010 may be a healthcare professional.

Sender 4000 may use a messaging modality 4001, which in this non-limiting example is SMS messages, to contact recipient 4010. Recipient 4010 may respond to such a contact or alternatively may choose to initiate contact. In either case, recipient 4010 may choose to initiate engagement through a chatbot 4007, for example to ask a question, request a chat conversation and/or to initiate an appointment. When the time comes for the appointment, or to book the appointment, recipient 4010 may initiate access through a recipient access point 4011, to reach an appointment booking module 4009. At the time of the appointment, sender 4000 and recipient 4010 may interact through a video module 4006 and/or a co-browsing module 4008. Load balancers 4002-4005 may help to support multi-modal communication between sender 4000 and recipient 4010.

FIG. 41 shows a non-limiting, exemplary backend system for communication through multiple modalities. As shown, a user interface 4100 preferably enables a user to access a client database 4101. In this non-limiting example, the user is an authorized user of an organization operating a system as described herein. For example, such an organization may comprise a HCP office or medical facility, and/or an external support staff organization. External support staff may include staff at diagnostic laboratories, other medical centers, and staff at providers of goods and services to healthcare professionals, including without limitation sales staff, pharmacists, medical device specialists, scientists and the like. Client database 4101 preferably stores information about the individuals and/or organizations served by the organization operating a system as described herein.

A configuration API 4102 may be used to configure a database 4106, which preferably stores information required for or in order to support a system as described herein. An authorization service 4103 may be used to determine authorized access to the backend system shown herein. Management of access to a particular aspect of the system as described herein may be controlled through authorization service 4103 and/or according to one or more policies determined through a client management application 4104 which may for example be determined by an admin application 4106. Required policy information may also be stored in database 4106.

A client admin application 4108 may contact a client self service application 4107, for example to adjust one or more parameters of the backend system, and optionally to store such adjustments in database 4106.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for supporting secure healthcare professional communication, comprising a computer network, a sending user computational device, a server and a recipient user computational device, wherein said sending user computational device, said server and said recipient user computational device communicate through said computer network;
wherein each of said sending user computational device and said recipient user computational device comprise a messaging modality, wherein said messaging modality comprises a modality addressed through a telephone number and a social media modality;
wherein said sending user computational device creates a message according to a template determined according to at least one policy; wherein said message is sent from said sending user computational device to said server and is addressed according to said telephone number or said social media address; and wherein said server relays said message according to said telephone number or said address to said recipient user computational device; wherein said telephone number or said address of said sending user computational device and said recipient user computational device are each masked.

2. The system of claim 1, wherein said sending user computational device comprises an external support staff user computational device and said recipient user computational device comprises a healthcare professional computational device, wherein the device is controllable by a healthcare professional.

3. The system of claim 2, wherein said external support staff comprises staff at diagnostic laboratories, other medical centers, and staff at providers of goods and services to healthcare professionals, including without limitation sales staff, pharmacists, medical device specialists and scientists; wherein said sending user computational device is associated with said external support staff and wherein said policy is enforced according to an external support staff organization.

4. The system of claim 1, wherein said telephone number is associated with a mobile device connected to or identified with an external support staff member or an external support staff organization.

5. The system of claim 1, wherein said telephone number is associated with a mobile device connected to or identified with a healthcare professional or a healthcare professional office.

6. The system of claim 5, wherein said message comprises an SMS message or a MMS message.

7. The system of claim 1, wherein said social media address comprises one or more of Facebook Messenger, WhatsApp, WeChat, iMessage, Apple Business Chat, Telegram, Signal, or Skype; and wherein said message is sent and/or received through said social media channel.

8. A method for increasing reliability of messaging communication through a messaging modality with a HCP (healthcare provider), wherein the messaging modality is executed through a computational device by the HCP, the method comprising: receiving a request for medical information through the messaging modality;
generating, under control of one or more computer systems, a message that provides the medical information and that includes message content, and recipient identification data for the HCP;
reviewing the message content for compliance by a computer system;
sending the message to the HCP if compliance has been established;
confirming a delivery of the message to the HCP; and
generating an audit trail that is configured to create a historical record corresponding to said message.

9. The method of claim 8, wherein said reviewing the message content for compliance comprises analyzing the message content by a NLP algorithm and preventing the message from being sent if non-compliant content is detected.

10. The method of claim 9, wherein said non-compliant content comprises one or more stop words.

11. The method of claim 9, wherein said non-compliant content comprises content that is not appropriate for the HCP, according to an analysis of a license or other certification held by the HCP.

12. The method of claim 9, further comprising confirming a delivery of the message to the HCP; or alternatively, upon receiving indication of delivery failure of the message, sending the message via a different redundant communication modality.

13. The method of claim 9, further comprising validating the message and/or a response from the HCP according to a contact detail and/or a parameter of the HCP.

14. The method of claim 13, wherein said parameter comprises one or both of a license of the HCP or an office address corresponding to a license of the HCP.

15. The method of claim 14, wherein the audit trail includes message timestamp data for the message, delivery attempt data including corresponding timestamp data, response data, identification data for the HCP and/or user identification data.

16. The method of claim 9, wherein transmitting the message through the messaging modality and the review for non-compliant content are compliant with healthcare and/or pharmaceutical service specific requirements when operated in combination.

17. A method for increasing reliability of messaging communication through a messaging modality with a patient, wherein the messaging modality is executed through a computational device by the patient, the method comprising: initiating transmission of patient-specific medical information through the messaging modality to the computational device by one or more of an HCP, HCP staff member and/or external support staff member;
generating, under control of one or more computer systems, a message that provides the patient specific medical information and that includes message content, and recipient identification data for the patient;
reviewing the message content for compliance by a computer system;
sending the message to the patient if compliance has been established;
confirming a delivery of the message to the patient; and
generating an audit trail that is configured to create a historical record corresponding to said message.

18. The method of claim 17, wherein said reviewing the message content for compliance comprises analyzing the message content by a NLP algorithm and preventing the message from being sent if non-compliant content is detected.

19. The method of claim 18, wherein said non-compliant content comprises one or more stop words.

20. The method of claim 18, wherein said non-compliant content comprises content that is not medically relevant to the patient.

21. The method of claim 18, wherein said non-compliant content comprises content that includes patient specific medical details that are protected under medical regulations.

22. The method of claim 18, wherein affirmative consent for such content to be transmitted through the messaging modality has not been provided by the patient.

23. The method of claim 18, further comprising confirming a delivery of the message to the patient; or alternatively, upon receiving indication of delivery failure of the message, sending the message via a different redundant communication modality.

24. The method of claim 18, further comprising validating the message and/or a response from the patient according to a contact detail of the patient.

25. The method of claim 24, wherein the audit trail includes message timestamp data for the message, delivery attempt data including corresponding timestamp data, response data, identification data for the patient and/or user identification data for the transmitting user.

26. The method of claim 18, wherein transmitting the message through the messaging modality and the review for non-compliant content are compliant with healthcare and/or pharmaceutical service specific requirements when operated in combination.
